(19) United States
(12) Patent Application Publication
(10) Pub. No.: US 2024/0423995 A1
(43) Pub. Date: Dec. 26, 2024

(54) C-KIT INHIBITORS AND USES FOR TREATING AND PREVENTING INFLAMMATORY CONDITIONS

(71) Applicant: GEORGETOWN UNIVERSITY, Washington, DC (US)

(72) Inventor: Charbel Moussa, Germantown, MD (US)

(73) Assignee: GEORGETOWN UNIVERSITY, Washington, DC (US)

(21) Appl. No.: 18/743,830

(22) Filed: Jun. 14, 2024

Related U.S. Application Data
(60) Provisional application No. 63/508,437, filed on Jun. 15, 2023.

(51) Int. Cl. A61K 31/5377; A61K 31/4365; A61K 45/06; A61P 29/00; A61P 37/06

(52) U.S. Cl. CPC A61K 31/5377; A61K 31/4365; A61K 45/06; A61P 29/00; A61P 37/06

(57) ABSTRACT

Disclosed are small molecule c-kit inhibitors useful in reducing or eliminating mast cell mediated inflammation. Pharmaceutical formulations containing the c-kit inhibitors are also disclosed. Additionally, methods of treating or preventing a condition, disorder, or disease using the c-kit inhibitors or pharmaceutical formulations thereof are disclosed. The condition, disorder, or disease may be an inflammatory condition, including flares of the inflammatory condition. Exemplary inflammatory conditions relevant to this disclosure include, but are not limited to, mastocytosis, mast cell activation syndrome, hereditary alpha tryptasemia, urticaria, Lyme disease, mast cell leukemia, chronic obstructive pulmonary disease, long COVID, asthma, inflammatory bowel disease, arthritis, allergy, and gout.

### PRIOR RELATED APPLICATION

This application claims the benefit of and priority to U.S. Provisional Application No. 63/508,437, filed on Jun. 15, 2023, which is hereby incorporated by reference in its entirety.

### TECHNICAL FIELD

The present disclosure relates to c-kit inhibitors. It also relates to pharmaceutical formulations of the c-kit inhibitors as well as methods for treating or preventing conditions, disorders, or diseases using the c-kit inhibitors.

### BACKGROUND

Mast cells are white blood cells that play a critical role in the innate immune system. Mast cells are formed in the bone marrow, circulate in the blood as immature (dormant) immune defense cells, and, in response to foreign antigens, the mast cells mature. Upon maturation, they migrate to connective tissue spaces and degranulate to release inflammatory factors, thereby triggering inflammation as part of the non-specific immune responses.

The tyrosine kinase c-kit (CD117) is expressed on the surface of mast cells. Activation of c-kit leads to activation of mast cells and their subsequent release of inflammatory factors. Certain human diseases like mast cell leukemia, mastocytosis, urticaria, and Lyme disease are related to c-kit activation. Allergic reaction is a condition driven by activation of mast cells in response to environmental stimuli, e.g., pollen, dust, food, and the like. These diseases and conditions are characterized by an overabundance of mature mast cells in the blood and/or tissues that triggers prolonged or permanent immune responses.

There is an urgent need for pharmacological treatments for inflammatory conditions as well as preventative measures for preventing or reducing flares in inflammatory conditions. The newly identified c-kit inhibitors described herein reduced the number of mature mast cell number or increase the ratio of immature mast cells to mature mast cells in a tissue, organ, or system of subjects who suffer from inflammatory conditions related to an overabundance of mature mast cells.

### SUMMARY

The present disclosure describes small molecule compounds that are newly identified as c-kit inhibitors. In general, the compounds have a structure of Formula I or a pharmaceutically acceptable salt, hydrate, or hydrated salt of Formula I,

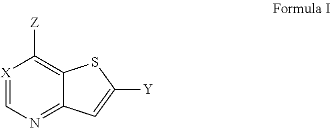

wherein X is N or CH;
wherein Y is C₆₋₁₀ aryl unsubstituted or substituted with R¹, C₅₋₁₀ heteroaryl unsubstituted or substituted with R¹, or N-methylpiperazinyl;
wherein R¹ is —CF₃, —(CH₂)ₙ—R², —(CH₂)ₙ—C(O)—R², or —O(CH₂)ₙ—R²;
wherein R² is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl;
wherein Z is heteroaryl, heterocyclyl, or NR³R⁴;
wherein R³ and R⁴ are independently H, C₁₋₃ alkyl, C₁₋₃ alkoxy, or phenyl unsubstituted or substituted with R¹⁰;
wherein R¹⁰ is halogen, —CN, hydroxyl, —CF₃, C₁₋₃ alkyl, C₁₋₃ alkoxy, amino, C₁₋₃ alkyl amino, or di C₁₋₃ alkyl amino; and
wherein n is an integer selected from 0 to 3.

Optionally, X is CH.

Optionally, Y is phenyl substituted with R¹.

Optionally, R¹ is —(CH₂)ₙ—R².

Optionally, R² is H.

Optionally, n is 1.

Optionally, Z is heterocyclyl, such as morpholin-1-yl.

Optionally, Z is NR³R⁴, such as NHR⁴. In some embodiments, R⁴ is phenyl unsubstituted or substituted with R¹⁰. In some embodiments, R⁴ is phenyl. In some embodiments, R⁴ is phenyl substituted with R¹⁰, wherein R¹⁰ is hydroxyl or methoxy.

Optionally, X is CH, Y is phenyl substituted with R¹, and Z is heterocyclyl. In some embodiments, Y is phenyl substituted with R¹ in the meta position and Z is morpholin-1-yl. In some embodiments, R¹ is —CH₃.

Optionally, X is CH, Y is phenyl substituted with R¹, and Z is NR³R⁴. In some embodiments, Y is phenyl substituted with R¹ in the meta position and Z is NHR⁴. In some embodiments, R¹ is —CH₃ and R⁴ is phenyl, 3-hydroxyphenyl, or 3-methoxyphenyl.

Exemplary compounds of this disclosure include, but are not limited to:

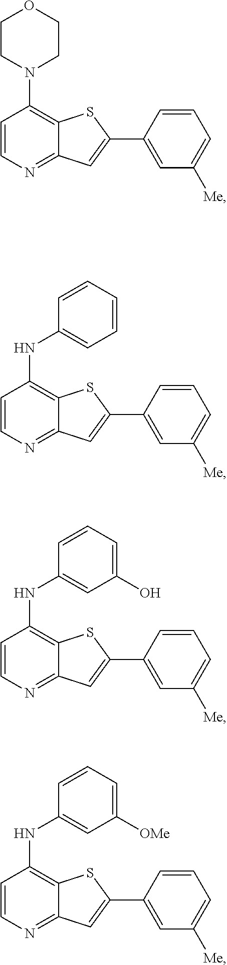 [SMILES: Cc1cccc(-c2cc3nccc(N4CCOCC4)c3s2)c1 ; Cc1cccc(-c2cc3nccc(Nc4ccccc4)c3s2)c1 ; Cc1cccc(-c2cc3nccc(Nc4cccc(O)c4)c3s2)c1 ; COc1cccc(Nc2ccnc3cc(-c4cccc(C)c4)sc23)c1]

and pharmaceutically acceptable salts, hydrates, and hydrated salts thereof.

Also disclosed are compositions containing a compound described herein. For example, the compositions may be a mixture of a salt form and a non-salt form of the compound.

Also disclosed are pharmaceutical formulations of the disclosed compounds or compositions. In general, the pharmaceutical formulations contain a pharmaceutically acceptable excipient. In some embodiments, the pharmaceutical formulations are in a form chosen from tablets, capsules, caplets, pills, beads, granules, particles, powders, gels, creams, solutions, suspensions, emulsions, sprays, inhalants, and nanoparticulate formulations. In some embodiments, the pharmaceutical formulations are oral formulations. In some embodiments, the pharmaceutical formulations are intravenous formulations. In some embodiments, the pharmaceutical formulations are intramuscular formulations. In some embodiments, the pharmaceutical formulations are topical formulations. In some embodiments, the pharmaceutical formulations are intranasal formulations. In some embodiments, the pharmaceutical formulations are inhalation formulations.

This disclosure also relates to (1) the compounds, compositions, and pharmaceutical formulations disclosed herein for treatment or prevention of an inflammatory condition, disorder, or disease disclosed herein or use as a medicament, (2) the compounds, compositions, and pharmaceutical formulations disclosed herein for use in the treatment or prevention of an inflammatory condition, disorder, or disease disclosed herein, or (3) the compounds, compositions, and pharmaceutical formulations disclosed herein for the manufacture of a medicament for treatment or prevention of an inflammatory condition, disorder, or disease disclosed herein.

Provided herein are methods of treating or preventing an inflammatory condition, disorder, or disease in a subject in need thereof. The method includes administering an effective amount of a compound, composition, or pharmaceutical formulation disclosed herein to the subject. In some embodiments, the compound, composition, or pharmaceutical formulation is administered systemically, locally, or by inhalation. In some embodiments, the effective amount of the compound, composition, or pharmaceutical formulation inhibits c-kit. In some embodiments, the effective amount of the compound, composition, or pharmaceutical formulation decreases mature mast cell number or increases a ratio of immature mast cells to mature mast cells in a tissue, organ, or system of the subject, as compared to the corresponding number or ratio before administrating or in the absence of the effective amount of the compound, composition, or pharmaceutical formulation.

In some embodiments, the inflammatory condition is present in a tissue, organ, or system of the subject, such as, by way of example, skin, connective tissue (e.g., cartilage or bone), mucosal tissue, joint, organ (e.g., intestines, lung, airways, spleen, pancreas), cardiovascular system, lymphatic system, skeletal system, respiratory system, and digestive system. In some embodiments, the inflammatory condition is related to c-kit upregulation or hyperactivity. In some embodiments, the inflammatory condition is related to an overabundance of mature mast cells.

Exemplary inflammatory conditions relevant to this disclosure include, but are not limited to, mastocytosis, mast cell activation syndrome, hereditary alpha tryptasemia, urticaria, Lyme disease, mast cell leukemia, chronic obstructive pulmonary disease, long COVID, asthma, inflammatory bowel disease, arthritis, allergy, and gout.

### BRIEF DESCRIPTION OF DRAWINGS

The present application includes the following drawings. The drawings are intended to illustrate certain embodiments and features or supplement any descriptions of the compounds, compositions, pharmaceutical formulations, and methods. The drawings do not limit the scope of the compounds, compositions, pharmaceutical formulations, and methods, unless the written description expressly indicates otherwise.

FIG. 1A is a bar graph showing the ratio of immature mast cells (MCs) to mature MCs in the absence and presence of varied concentrations (10 nM, 100 nM, and 1 μM) of BK40413. The ratio of immature MCs to mature MCs was represented by the ratio of MCs expressing CD117 only (CD117+) to MCs expressing both CD117 and FceR1 (CD117+/FceR1+). 1 μM p=0.03, 100 nM p=0.01, one-tailed Student's t-test.

FIG. 1B is a bar graph showing the ratio of immature MCs to mature MCs in the absence and presence of varied concentrations (10 nM, 100 nM, and 1 μM) of BK40197. The ratio of immature MCs to mature MCs was represented by the ratio of MCs expressing CD117 only (CD117+) to MCs expressing both CD117 and FceR1 (CD117+/FceR1+). 1 μM p=0.03, 100 nM p=0.01, one-tailed Student's t-test.

FIG. 1C is a bar graph showing the ratio of immature MCs to mature MCs in the absence and presence of varied concentrations (10 nM, 100 nM, and 1 μM) of BK40196. The ratio of immature MCs to mature MCs was represented by the ratio of MCs expressing CD117 only (CD117+) to MCs expressing both CD117 and FceR1 (CD117+/FceR1+). 10 nM p=0.03, one-tailed Student's t-test.

FIG. 2 is a bar graph showing the flow cytometry gating for mature peripheral MCs from blood samples of wild-type C57BL/6 mice treated by vehicle (DMSO) or BK40197 (at 50 mg/kg per day) for five days. The population of mature peripheral MCs correspond to the CD45+/FceR1+/CD117+ cell population.

FIG. 3 is a bar graph showing the ratio of immature MCs to mature MCs from blood samples of wild-type C57BL/6 mice (the “WT” bar), transgenic amyloid precursor protein (APP) mice (the “TgAPP” bar), A53T alpha-synuclein transgenic mice (the “A53T” bar), and tau transgenic 4510 mice (the “Tg4510” bar). The ratio of immature MCs to mature MCs was represented by the ratio of MCs expressing CD117 only (CD117+) to MCs expressing both CD117 and FceR1 (CD117+/FceR1+). p=0.08, ordinary one-way ANOVA.

FIG. 4 is a bar graph showing the ratio of immature MCs to mature MCs from blood samples of transgenic APP mice treated by vehicle (DMSO) or BK40143 (at 5 mg/kg per day) for six weeks. The ratio of immature MCs to mature MCs was represented by the ratio of MCs expressing CD117 only (CD117+) to MCs expressing both CD117 and FceR1 (CD117+/FceR1+). p=0.03, two-tailed Student's t-test.

FIG. 5 is a bar graph showing the ratio of immature MCs to mature MCs from blood samples of transgenic APP mice treated by vehicle (DMSO) or BK40195 (at 25 mg/kg per day) for six weeks. The ratio of immature MCs to mature MCs was represented by the ratio of MCs expressing CD117 only (CD117+) to MCs expressing both CD117 and FceR1 (CD117+/FceR1+). p=0.04, one-tailed Student's t-test.

FIG. 6 is a bar graph showing the ratio of immature MCs to mature MCs from blood samples of transgenic APP mice treated by vehicle (DMSO) or BK40197 (at 45 mg/kg per day) for six weeks. The ratio of immature MCs to mature MCs was represented by the ratio of MCs expressing CD117 only (CD117+) to MCs expressing both CD117 and FceR1 (CD117+/FceR1+). p=0.07, one-tailed Student's t-test.

FIG. 7A is a bar graph showing the flow cytometry gating for MCs from spleen samples of transgenic APP mice treated by vehicle (DMSO), BK40196 at 20 mg/kg per day, or BK40196 at 40 mg/kg per day for six weeks. The population of MCs correspond to the FceR1+ cell population. 20 mg/kg, p=0.01; 40 mg/kg p=0.006, one-tailed Student's t-test.

FIG. 7B is a bar graph showing the flow cytometry gating for mature MCs from spleen samples of transgenic APP mice treated by vehicle (DMSO), BK40196 at 20 mg/kg per day, or BK40196 at 40 mg/kg per day for six weeks. The population of mature MCs correspond to the CD117+/FceR1+ cell population. p=0.02, one-tailed Student's t-test.

FIG. 7C is a bar graph showing the ratio of immature MCs to mature MCs from spleen samples of transgenic APP mice treated by vehicle (DMSO), BK40196 at 20 mg/kg per day, or BK40196 at 40 mg/kg per day for six weeks. The ratio of immature MCs to mature MCs was represented by the ratio of MCs expressing CD117 only (CD117+) to MCs expressing both CD117 and FceR1 (CD117+/FceR1+). p=0.03, one-tailed Student's t-test.

FIG. 8A shows a toluidine blue stain of blood smear from male and female, 4-8 month old wild type C57BL/6 mice, treated only once with intraperitoneal (I.P.) injection of DMSO (control). This figure shows mast cells 3-4 hours post-injection.

FIG. 8B shows a toluidine blue stain of blood smear from male and female, 4-8 month old wild type C57BL/6 mice, injected with DMSO approximately 2 hours prior to injection (IP) with 50 μg C48/40. This figure shows degranulation of mast cells 0.5-1.5 hours post-injection of C48/40.

FIG. 8C shows a toluidine blue stain of blood smear from male and female, 4-8 month old wild type C57BL/6 mice, injected with 20 mg/kg BK40197, approximately two hours before I.P. injection of C48/40. This figure shows the absence of mast cell degranulation approximately 0.5-1.5 hours post-injection of C48/40. n=6 per treatment.

FIG. 8D shows a toluidine blue stain of blood smear from male and female, 4-8 month old wild type C57BL/6 mice, injected with 20 mg/kg BK40195, approximately two hours before I.P. injection of C48/40. This figure shows the absence of mast cell degranulation approximately 0.5-1.5 hours post-injection of C48/40. n=6 per treatment.

FIG. 9A shows a toluidine blue stain of blood smear from male and female, 4-8 month old wild type C57BL/6 mice, treated only once with intraperitoneal (I.P.) injection of DMSO (control). This figure shows mast cells 3-4 hours post-injection.

FIG. 9B shows a toluidine blue stain of blood smear from male and female, 4-8 month old wild type C57BL/6 mice, injected with DMSO approximately 2 hours prior to injection (I.P) with 50 μg C48/40. This figure shows degranulation of mast cells 0.5-1.5 hours post-injection of C48/40.

FIG. 9C shows a toluidine blue stain of blood smear from male and female, 4-8 month old wild type C57BL/6 mice, injected with 10 mg/kg BK40143, approximately two hours before I.P. injection of C48/40. This figure shows the absence of mast cell degranulation approximately 0.5-1.5 hours post-injection of C48/40. n=6 per treatment.

FIG. 9D shows a toluidine blue stain of blood smear from male and female, 4-8 month old wild type C57BL/6 mice, injected with 10 mg/kg BK40196, approximately two hours before I.P. injection of C48/40. This figure shows the absence of mast cell degranulation approximately 0.5-1.5 hours post-injection of C48/40. n=6 per treatment.

### DETAILED DESCRIPTION

Provided herein are small molecule compounds that were newly discovered to be c-kit inhibitors. Also disclosed are compositions and pharmaceutical formulations containing a compound described herein. Additionally, methods of treating or preventing an inflammatory condition, disorder, or disease or conditions, disorders, or diseases associated with an overabundance of mature mast cells using the compounds, compositions, or pharmaceutical formulations are disclosed. Such conditions, disorders, or diseases include, but are not limited to, mastocytosis, mast cell activation syndrome, hereditary alpha tryptasemia, urticaria, Lyme disease, mast cell leukemia, chronic obstructive pulmonary disease, long COVID, asthma, inflammatory bowel disease, arthritis, allergy, and gout.

Before the present disclosure is described in greater detail, it is to be understood that this disclosure is not limited to the particular embodiments described herein and, as such, may vary in accordance with the scope of the present disclosure. All publications and patents cited in this specification are herein incorporated by reference as if each individual publication and patent were specifically and individually indicated to be incorporated by reference. They are incorporated by reference to disclose and describe the methods and/or materials in connection with which the publications and patents are cited.

As will be apparent to those of ordinary skill in the art upon reading this disclosure, each of the particular embodiments described and illustrated herein has discrete components and/or features that may be readily separated from or combined with one or more components and/or features of any of the other embodiments described herein, without departing from the scope or spirit of the present disclosure. Any recited method can be carried out in the order of events recited herein or in any other order that is logically possible.

### I. Compounds

The present disclosure describes small molecule compounds that are newly discovered to be effective c-kit inhibitors capable of reducing inflammation and/or reducing the number of mature mast cells or increases a ratio of immature mast cells to mature cells.

To the extent that chemical formulas described herein contain one or more unspecified chiral centers, the formulas are intended to encompass all stable stereoisomers, enantiomers, and diastereomers. Such compounds can exist as a single enantiomer, a racemic mixture, a mixture of diastereomers, or combinations thereof. It is also understood that the chemical formulas encompass all tautomeric forms if tautomerization occurs.

Methods of making exemplary compounds are disclosed in subsequent sections and exemplified by the Examples. The synthetic methods disclosed herein are compatible with a wide variety of functional groups and starting materials. Thus, a wide variety of compounds can be obtained from the disclosed methods.

Optionally, the alkyl groups described herein have 1-20 carbon atoms, i.e., C₁₋₂₀ alkyl. In some forms, the C₁₋₂₀ alkyl can be a linear C₁₋₂₀ alkyl or a branched C₃₋₂₀ alkyl. Optionally, the alkyl groups have 1-10 carbon atoms, i.e., C₁₋₁₀ alkyl. In some forms, the C₁₋₁₀ alkyl can be a linear C₁₋₁₀ alkyl or a branched C₃₋₁₀ alkyl. Optionally, the alkyl groups have 1-6 carbon atoms, i.e., C₁₋₆ alkyl. In some forms, the C₁₋₆ alkyl can be a linear C₁₋₆ alkyl or a branched C₃₋₆ alkyl. Optionally, the alkyl groups have 1-4 carbon atoms, i.e., C₁₋₄ alkyl. In some forms, the C₁₋₄ alkyl can be a linear C₁₋₄ alkyl or a branched C₃₋₄ alkyl. Representative straight chain alkyl groups include methyl, ethyl, n-propyl, n-butyl, n-pentyl, n-hexyl, n-heptyl, n-octyl, n-nonyl, and the like. Representative branched alkyl groups include isopropyl, sec-butyl, isobutyl, tert-butyl, isopentyl, and the like.

Optionally, the aryl groups described herein have 6-20 carbon atoms, i.e., C₆₋₂₀ aryl. Optionally, the aryl groups have 6-12 carbon atoms, i.e., C₆₋₁₂ aryl. Representative aryl groups include phenyl, naphthyl, and biphenyl.

Optionally, the heteroaryl groups have 1-20 carbon atoms, i.e., C₁₋₂₀ heteroaryl. Optionally, the heteroaryl groups have 1-11 carbon atoms, i.e., C₁₋₁₁ heteroaryl. Optionally, the heteroaryl groups have 1-5 carbon atoms, i.e., C₁₋₅ heteroaryl. Optionally, the heteroaryl groups are 5-20 membered heteroaryl groups. Optionally, the heteroaryl groups are 5-12 membered heteroaryl groups. Optionally, the heteroaryl groups are 5 or 6 membered heteroaryl groups. Exemplary heteroatoms in the heteroaryl groups include O, N, and S. Representative heteroaryl groups include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

Optionally, the heterocyclyl groups described herein have 1-20 carbon atoms, i.e., C₁₋₂₀ heterocyclyl. Optionally, the heterocyclyl groups described herein have 1-11 carbon atoms, i.e., C₁₋₁₁ heterocyclyl. Optionally, the heterocyclyl groups described herein have 1-6 carbon atoms, i.e., C₁₋₆ heterocyclyl. Optionally, the heterocyclyl groups are 3-20 membered heterocyclyl groups. Optionally, the heterocyclyl groups are 3-12 membered heterocyclyl groups. Optionally, the heteroaryl groups are 4-7 membered heterocyclyl groups.

As used herein, “alkoxy” or “alkyloxy” refers to a hydroxyl group substituted by an alkyl group at the oxygen atom. Exemplary alkyloxy groups include, but are not limited to, methoxy, ethoxy, n-propoxy, i-propoxy, n-butoxy, s-butoxy, t-butoxy, n-pentoxy, and s-pentoxy.

As used herein, “alkylamino” refers to an amino group (—NH₂) substituted by one or two alkyl groups. When the amino group is substituted by two alkyl groups, the two alkyl groups can be the same or different. An example of alkylamino is methylamino (—NH—CH₃).

### A. General Formula

In general, the compounds have a structure of Formula I or a pharmaceutically acceptable salt, hydrate, or hydrated salt of Formula I,

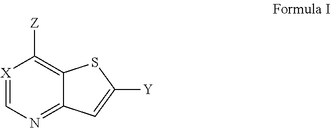

wherein X is N or CH;
wherein Y is C₆₋₁₀ aryl unsubstituted or substituted with R¹, C₅₋₁₀ heteroaryl unsubstituted or substituted with R¹, or N-methylpiperazinyl;
wherein R¹ is —CF₃, —(CH₂)ₙ—R², —(CH₂)ₙ—C(O)—R², or —O(CH₂)ₙ—R².
wherein R² is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl;
wherein Z is heteroaryl, heterocyclyl, or NR³R⁴;
wherein R³ and R⁴ are independently H, C₁₋₃ alkyl, C₁₋₃ alkoxy, or phenyl unsubstituted or substituted with R¹⁰;
wherein R¹⁰ is halogen, —CN, hydroxyl, —CF₃, C₁₋₃ alkyl, C₁₋₃ alkoxy, amino, C₁₋₃ alkyl amino, or di C₁₋₃ alkyl amino; and
wherein n is an integer selected from 0 to 3.

Optionally, X is CH. Optionally, X is N.

Optionally, the compounds do not contain any halogen atoms.

Optionally, the compounds are in a non-salt form as shown in Formula I. Optionally, the compounds are in a salt form. In some embodiments, the compounds are in an HCl salt form.

### 1. The Y Moiety

Optionally, Y is C₅₋₁₀ heteroaryl unsubstituted or substituted with R¹. Optionally, Y is N-methylpiperazinyl. Optionally, Y is C₆₋₁₀ aryl unsubstituted or substituted with R¹. In some embodiments, Y is phenyl. In some embodiments, Y is phenyl substituted with R¹. In some embodiments, Y is phenyl substituted with R¹ in the meta position.

Optionally, R¹ is —CF₃. Optionally, R¹ is —(CH₂)ₙ—R². Optionally, R¹ is —(CH₂)ₙ—C(O)—R². Optionally, R¹ is —O(CH₂)ₙ—R². Optionally, R² is H.

Optionally, R² is —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl. In some embodiments, R² is —CN, halogen, or amino.

Optionally, n is 0. Optionally, n is 1. Optionally, n is 2. Optionally, n is 3.

In some embodiments, R¹ is —CH₃ or —CF₃.

In some embodiments, Y is phenyl substituted with R¹, wherein R¹ is —(CH₂)ₙ—R². In some embodiments, R¹ is —CH₃ or —CF₃, i.e., Y is methylphenyl or (trifluoromethyl)phenyl, respectively.

In some embodiments, Y is phenyl substituted with R¹ in the meta position, wherein R¹ is —(CH₂)ₙ—R². In some embodiments, R¹ is —CH₃ or —CF₃, i.e., Y is 3-methylphenyl or 3-(trifluoromethyl)phenyl, respectively.

### 2. The Z Moiety

Optionally, Z is heteroaryl. Optionally, Z is heterocyclyl, such as morpholin-1-yl. Optionally, Z is NR³R⁴.

Optionally, R³ is H. Optionally, R³ is C₁₋₃ alkyl, such as methyl. Optionally, R³ is C₁₋₃ alkoxy. Optionally, R³ is phenyl unsubstituted or substituted with R¹⁰. In some embodiments, R³ is phenyl. In some embodiments, R³ is phenyl substituted with R¹⁰. In some embodiments, R³ is phenyl substituted with R¹⁰ at the meta position.

Optionally, R⁴ is H. Optionally, R⁴ is C₁₋₃ alkyl, such as methyl. Optionally, R⁴ is C₁₋₃ alkoxy. Optionally, R⁴ is phenyl unsubstituted or substituted with R¹⁰. In some embodiments, R⁴ is phenyl. In some embodiments, R⁴ is phenyl substituted with R¹⁰. In some embodiments, R⁴ is phenyl substituted with R¹⁰ at the meta position.

Optionally, R¹⁰ is hydroxyl. Optionally, R¹⁰ is C₁₋₃ alkoxy, such as methoxy. Optionally, R¹⁰ is halogen, —CN, —CF₃, C₁₋₃ alkyl, amino, C₁₋₃ alkyl amino, or di C₁₋₃ alkyl amino.

In some embodiments, R³ is phenyl, hydroxyphenyl, or methoxyphenyl. In some embodiments, R³ is phenyl, 3-hydroxyphenyl, or 3-methoxyphenyl.

In some embodiments, R⁴ is phenyl, hydroxyphenyl, or methoxyphenyl. In some embodiments, R⁴ is phenyl, 3-hydroxyphenyl, or 3-methoxyphenyl.

In some embodiments, Z is NHR⁴. In some embodiments, R⁴ is phenyl unsubstituted or substituted with R¹⁰. In some embodiments, R⁴ is phenyl. In some embodiments, R⁴ is phenyl substituted with R¹⁰. In some embodiments, R⁴ is hydroxyphenyl or methoxyphenyl. In some embodiments, R⁴ is phenyl substituted with R¹⁰ at the meta position. In some embodiments, R⁴ is 3-hydroxyphenyl or 3-methoxyphenyl.

In some embodiments, Z is NHR⁴ and R⁴ is phenyl, hydroxyphenyl, or methoxyphenyl.

In some embodiments, Z is NHR⁴ and R⁴ is phenyl, 3-hydroxyphenyl, or 3-methoxyphenyl.

### B. Sub-Formulas

In some embodiments, Y is phenyl, so Formula I is in the following form, wherein X and Z are the same as those described above.

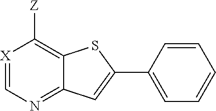

In some embodiments, Y is phenyl substituted with R¹, so Formula I is in the following form, wherein X, Z, and R¹ are the same as those described above.

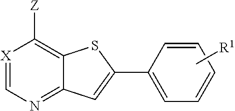

In some embodiments, Y is phenyl substituted with R¹ in the meta position, so Formula I is in the following form, wherein X, Z, and R¹ are the same as those described above.

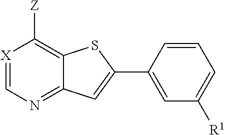

In some embodiments, Z is morpholin-1-yl, so Formula I is in the following form, wherein X and Y are the same as those described above.

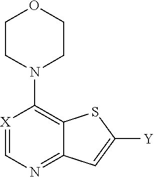

In some embodiments, Z is NHR⁴ and R⁴ is phenyl, so Formula I is in the following form, wherein X and Y are the same as those described above.

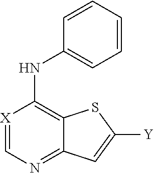

In some embodiments, Z is NHR⁴ and R⁴ is phenyl substituted with R¹⁰, so Formula I is in the following form, wherein X, Y, and R¹⁰ are the same as those described above.

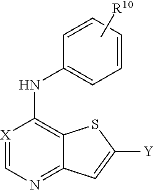

In some embodiments, Z is NHR⁴ and R⁴ is phenyl substituted with R¹⁰ at the meta position, so Formula I is in the following form, wherein X, Y, and R¹⁰ are the same as those described above.

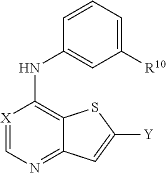

In some embodiments, Y is phenyl and Z is morpholin-1-yl, so Formula I is in the following form, wherein X is the same as that described above.

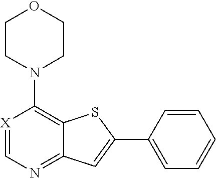

In some embodiments, Y is phenyl, Z is NHR⁴, and R⁴ is phenyl, so Formula I is in the following form, wherein X is the same as that described above.

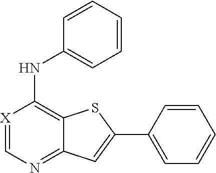

In some embodiments, Y is phenyl, Z is NHR⁴ and R⁴ is phenyl substituted with R¹⁰, so Formula I is in the following form, wherein X and R¹⁰ are the same as those described above.

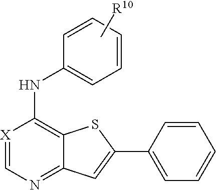

In some embodiments, Y is phenyl, Z is NHR⁴, and R⁴ is phenyl substituted with R¹⁰ at the meta position, so Formula I is in the following form, wherein X and R¹⁰ are the same as those described above.

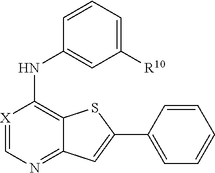

In some embodiments, Y is phenyl substituted with R¹ and Z is morpholin-1-yl, so Formula I is in the following form, wherein X and R¹ are the same as those described above.

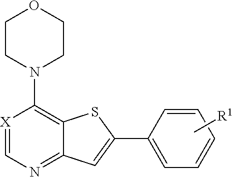

In some embodiments, Y is phenyl substituted with R¹, Z is NHR⁴, and R⁴ is phenyl, so Formula I is in the following form, wherein X and R¹ are the same as those described above.

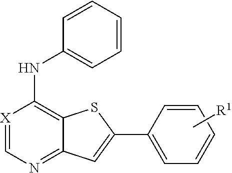

In some embodiments, Y is phenyl substituted with R¹, Z is NHR⁴, and R⁴ is phenyl substituted with R¹⁰, so Formula I is in the following form, wherein X, R¹, and R¹⁰ are the same as those described above.

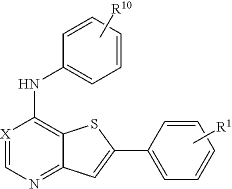

In some embodiments, Y is phenyl substituted with R¹, Z is NHR⁴, and R⁴ is phenyl substituted with R¹⁰ at the meta position, so Formula I is in the following form, wherein X, R¹, and R¹⁰ are the same as those described above.

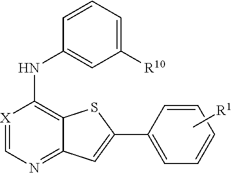

In some embodiments, Y is phenyl substituted with R¹ at the meta position and Z is morpholin-1-yl, so Formula I is in the following form, wherein X and R¹ are the same as those described above.

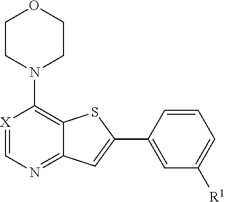

In some embodiments, Y is phenyl substituted with R¹ at the meta position, Z is NHR⁴, and R⁴ is phenyl, so Formula I is in the following form, wherein X and R¹ are the same as those described above.

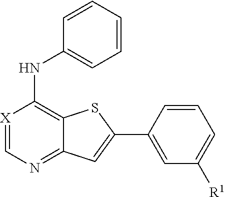

In some embodiments, Y is phenyl substituted with R¹ at the meta position, Z is NHR⁴, and R⁴ is phenyl substituted with R¹⁰, so Formula I is in the following form, wherein X, R¹, and R¹⁰ are the same as those described above.

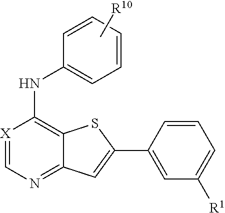

In some embodiments, Y is phenyl substituted with R¹ at the meta position, Z is NHR⁴, and R⁴ is phenyl substituted with R¹⁰ at the meta position, so Formula I is in the following form, wherein X, R¹, and R¹⁰ are the same as those described above.

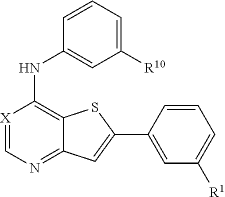

Optionally, the compounds of the foregoing sub-formulas do not contain any halogen atoms.

Optionally, the compounds of the foregoing sub-formulas are in a non-salt form as shown in the sub-formulas. Optionally, the compounds are in a salt form. In some embodiments, the compounds are in an HCl salt form.

### C. Exemplary Structures

Optionally, X is CH, Y is phenyl, and Z is heterocyclyl. In some embodiments, Z is morpholin-1-yl.

Optionally, X is CH, Y is phenyl, and Z is NR³R⁴. In some embodiments, Z is NHR⁴. In some embodiments, R⁴ is phenyl unsubstituted or substituted with R¹⁰. In some embodiments, R⁴ is phenyl. In some embodiments, R⁴ is phenyl substituted with R¹⁰. In some embodiments, R⁴ is hydroxyphenyl or methoxyphenyl. In some embodiments, R⁴ is phenyl substituted with R¹⁰ at the meta position. In some embodiments, R⁴ is 3-hydroxyphenyl or 3-methoxyphenyl. In some embodiments, R⁴ is selected from phenyl, hydroxyphenyl, and methoxyphenyl. In some embodiments, R⁴ is selected from phenyl, 3-hydroxyphenyl, and 3-methoxyphenyl.

Optionally, X is CH, Y is phenyl substituted with R¹, and Z is heterocyclyl. In some embodiments, Y is phenyl substituted with R¹, wherein R¹ is —(CH₂)ₙ—R²; for example, R¹ is —CH₃ or —CF₃, i.e., Y is methylphenyl or (trifluoromethyl)phenyl, respectively. In some embodiments, Y is phenyl substituted with R¹ in the meta position, wherein R¹ is —(CH₂)ₙ—R²; for example, R¹ is —CH₃ or —CF₃, i.e., Y is 3-methylphenyl or 3-(trifluoromethyl)phenyl, respectively. In some embodiments, Z is morpholin-1-yl. In some embodiments, Y is phenyl substituted with R¹ in the meta position and Z is morpholin-1-yl. In some embodiments, Y is phenyl substituted with R¹ in the meta position, Z is morpholin-1-yl, and R¹ is —CH₃ or —CF₃. In some embodiments, Y is phenyl substituted with R¹ in the meta position, Z is morpholin-1-yl, and R¹ is —CH₃.

Optionally, X is CH, Y is phenyl substituted with R¹, and Z is NR³R⁴. In some embodiments, Y is phenyl substituted with R¹, wherein R¹ is —(CH₂)ₙ—R²; for example, R¹ is —CH₃ or —CF₃, i.e., Y is methylphenyl or (trifluoromethyl)phenyl, respectively. In some embodiments, Y is phenyl substituted with R¹ in the meta position, wherein R¹ is —(CH₂)ₙ—R²; for example, R¹ is —CH₃ or —CF₃, i.e., Y is 3-methylphenyl or 3-(trifluoromethyl)phenyl, respectively. In some embodiments, Z is NHR⁴. In some embodiments, R⁴ is phenyl unsubstituted or substituted with R¹⁰. In some embodiments, R⁴ is phenyl. In some embodiments, R⁴ is phenyl substituted with R¹⁰. In some embodiments, R⁴ is hydroxyphenyl or methoxyphenyl. In some embodiments, R⁴ is phenyl substituted with R¹⁰ at the meta position. In some embodiments, R⁴ is 3-hydroxyphenyl or 3-methoxyphenyl. In some embodiments, R⁴ is selected from phenyl, hydroxyphenyl, and methoxyphenyl. In some embodiments, R⁴ is selected from phenyl, 3-hydroxyphenyl, and 3-methoxyphenyl. In some embodiments, Y is phenyl substituted with R¹ in the meta position and Z is NHR⁴. In some embodiments, Y is phenyl substituted with R¹ in the meta position, Z is NHR⁴, R¹ is —CH₃ or —CF₃, and R⁴ is phenyl, hydroxyphenyl, or methoxyphenyl. In some embodiments, Y is phenyl substituted with R¹ in the meta position, Z is NHR⁴, R¹ is —CH₃, and R⁴ is phenyl, 3-hydroxyphenyl, or 3-methoxyphenyl.

Exemplary compounds include, but are not limited to, the following:

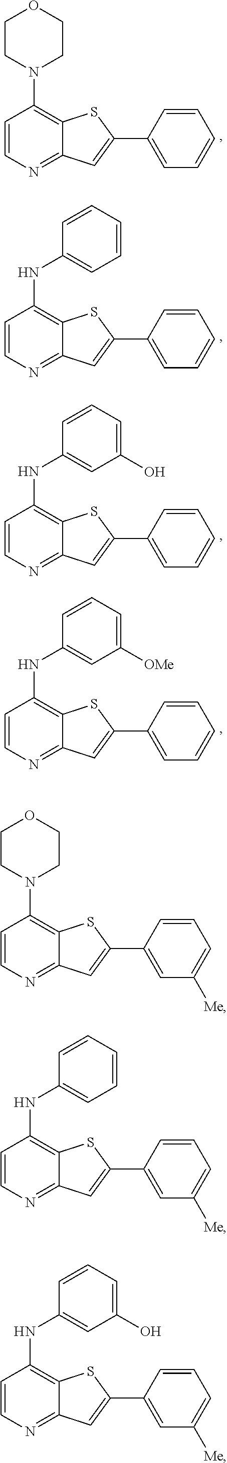

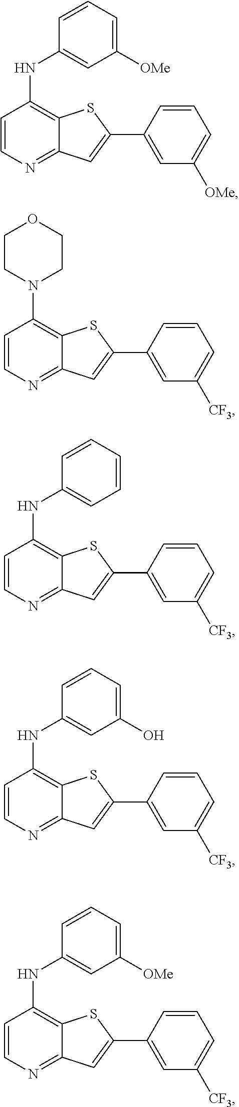 [SMILES: COc1cccc(Nc2ccnc3cc(-c4cccc(OC)c4)sc23)c1 ; FC(F)(F)c1cccc(-c2cc3nccc(N4CCOCC4)c3s2)c1 ; FC(F)(F)c1cccc(-c2cc3nccc(Nc4ccccc4)c3s2)c1 ; Oc1cccc(Nc2ccnc3cc(-c4cccc(C(F)(F)F)c4)sc23)c1 ; COc1cccc(Nc2ccnc3cc(-c4cccc(C(F)(F)F)c4)sc23)c1]

and pharmaceutically acceptable salts, hydrates, and hydrated salts thereof.

In some embodiments, the compounds are selected from:

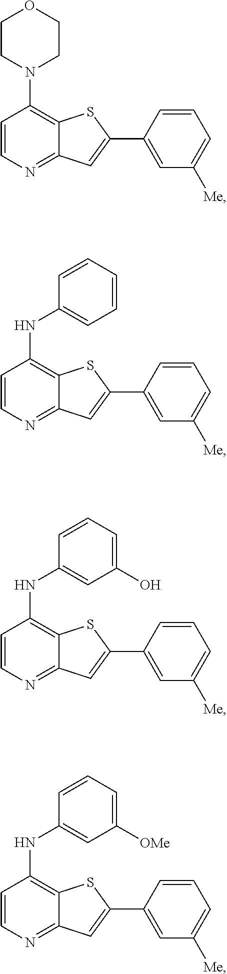 [SMILES: Cc1cccc(-c2cc3nccc(N4CCOCC4)c3s2)c1 ; Cc1cccc(-c2cc3nccc(Nc4ccccc4)c3s2)c1 ; Cc1cccc(-c2cc3nccc(Nc4cccc(O)c4)c3s2)c1 ; COc1cccc(Nc2ccnc3cc(-c4cccc(C)c4)sc23)c1]

and pharmaceutically acceptable salts, hydrates, and hydrated salts thereof.

Optionally, X is N, Y is phenyl, and Z is heterocyclyl. In some embodiments, Z is morpholin-1-yl.

Optionally, X is N, Y is phenyl, and Z is NR³R⁴. In some embodiments, Z is NHR⁴. In some embodiments, R⁴ is phenyl unsubstituted or substituted with R¹⁰. In some embodiments, R⁴ is phenyl. In some embodiments, R⁴ is phenyl substituted with R¹⁰. In some embodiments, R⁴ is hydroxyphenyl or methoxyphenyl. In some embodiments, R⁴ is phenyl substituted with R¹⁰ at the meta position. In some embodiments, R⁴ is 3-hydroxyphenyl or 3-methoxyphenyl. In some embodiments, R⁴ is selected from phenyl, hydroxyphenyl, and methoxyphenyl. In some embodiments, R⁴ is selected from phenyl, 3-hydroxyphenyl, and 3-methoxyphenyl.

Optionally, X is N, Y is phenyl substituted with R¹, and Z is heterocyclyl. In some embodiments, Y is phenyl substituted with R¹, wherein R¹ is —(CH₂)ₙ—R²; for example, R¹ is —CH₃ or —CF₃, i.e., Y is methylphenyl or (trifluoromethyl)phenyl, respectively. In some embodiments, Y is phenyl substituted with R¹ in the meta position, wherein R¹ is —(CH₂)ₙ—R²; for example, R¹ is —CH₃ or —CF₃, i.e., Y is 3-methylphenyl or 3-(trifluoromethyl)phenyl, respectively. In some embodiments, Z is morpholin-1-yl. In some embodiments, Y is phenyl substituted with R¹ in the meta position and Z is morpholin-1-yl. In some embodiments, Y is phenyl substituted with R¹ in the meta position, Z is morpholin-1-yl, and R¹ is —CH₃ or —CF₃. In some embodiments, Y is phenyl substituted with R¹ in the meta position, Z is morpholin-1-yl, and R¹ is —CH₃.

Optionally, X is N, Y is phenyl substituted with R¹, and Z is NR³R⁴. In some embodiments, Y is phenyl substituted with R¹, wherein R¹ is —(CH₂)ₙ—R²; for example, R¹ is —CH₃ or —CF₃, i.e., Y is methylphenyl or (trifluoromethyl)phenyl, respectively. In some embodiments, Y is phenyl substituted with R¹ in the meta position, wherein R¹ is —(CH₂)ₙ—R²; for example, R¹ is —CH₃ or —CF₃, i.e., Y is 3-methylphenyl or 3-(trifluoromethyl)phenyl, respectively. In some embodiments, Z is NHR⁴. In some embodiments, R⁴ is phenyl unsubstituted or substituted with R¹⁰. In some embodiments, R⁴ is phenyl. In some embodiments, R⁴ is phenyl substituted with R¹⁰. In some embodiments, R⁴ is hydroxyphenyl or methoxyphenyl. In some embodiments, R⁴ is phenyl substituted with R¹⁰ at the meta position. In some embodiments, R⁴ is 3-hydroxyphenyl or 3-methoxyphenyl. In some embodiments, R⁴ is selected from phenyl, hydroxyphenyl, and methoxyphenyl. In some embodiments, R⁴ is selected from phenyl, 3-hydroxyphenyl, and 3-methoxyphenyl. In some embodiments, Y is phenyl substituted with R¹ in the meta position and Z is NHR⁴. In some embodiments, Y is phenyl substituted with R¹ in the meta position, Z is NHR⁴, R¹ is —CH₃ or —CF₃, and R⁴ is phenyl, hydroxyphenyl, or methoxyphenyl. In some embodiments, Y is phenyl substituted with R¹ in the meta position, Z is NHR⁴, R¹ is —CH₃, and R⁴ is phenyl, 3-hydroxyphenyl, or 3-methoxyphenyl.

Exemplary compounds include, but are not limited to, the following:

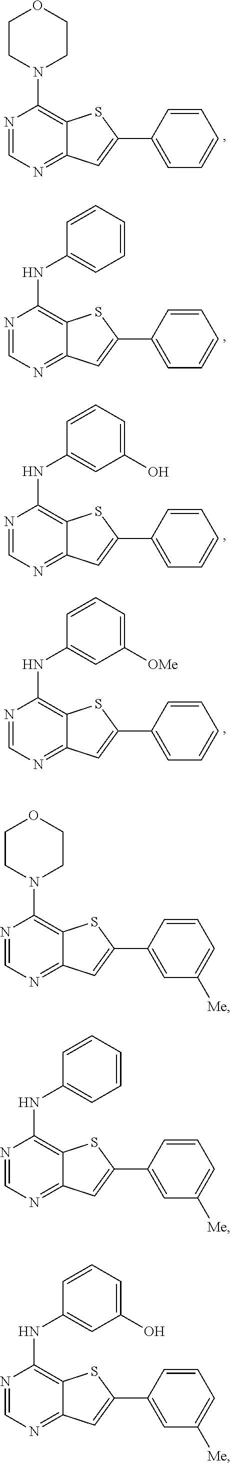

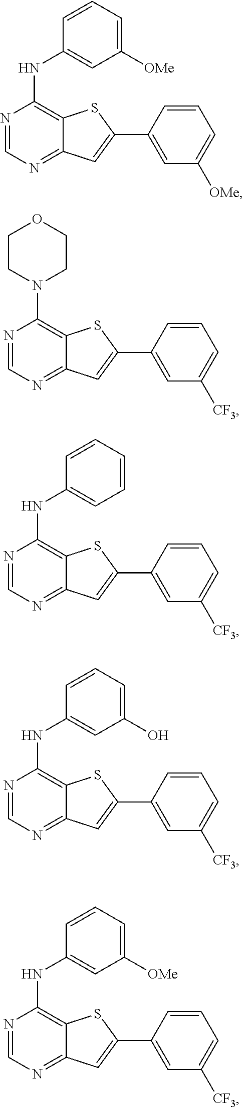 [SMILES: COc1cccc(Nc2ncnc3cc(-c4cccc(OC)c4)sc23)c1 ; FC(F)(F)c1cccc(-c2cc3ncnc(N4CCOCC4)c3s2)c1 ; FC(F)(F)c1cccc(-c2cc3ncnc(Nc4ccccc4)c3s2)c1 ; Oc1cccc(Nc2ncnc3cc(-c4cccc(C(F)(F)F)c4)sc23)c1 ; COc1cccc(Nc2ncnc3cc(-c4cccc(C(F)(F)F)c4)sc23)c1]

and pharmaceutically acceptable salts, hydrates, and hydrated salts thereof.

In some embodiments, the compounds are selected from:

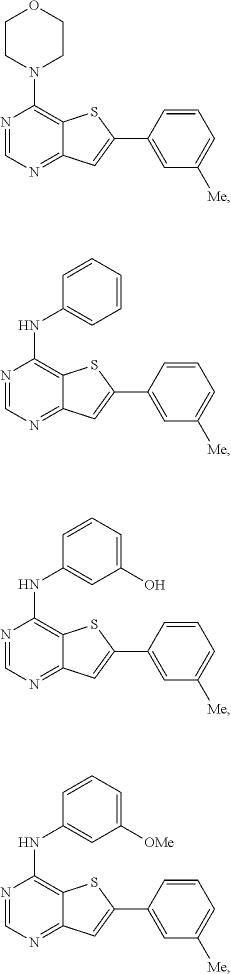 [SMILES: Cc1cccc(-c2cc3ncnc(N4CCOCC4)c3s2)c1 ; Cc1cccc(-c2cc3ncnc(Nc4ccccc4)c3s2)c1 ; Cc1cccc(-c2cc3ncnc(Nc4cccc(O)c4)c3s2)c1 ; COc1cccc(Nc2ncnc3cc(-c4cccc(C)c4)sc23)c1]

and pharmaceutically acceptable salts, hydrates, and hydrated salts thereof.

### II. Compositions

The disclosed compounds may be present in a mixture of a salt form and a non-salt form. In some embodiments, more than 50%, 60%, 70%, 80%, 90%, 95%, or 98% of the compound in the mixture may be in the non-salt form, calculated as the ratio of the weight of the non-salt form to the total weight of the mixture. In some embodiments, more than 50%, 60%, 70%, 80%, 90%, 95%, or 98% of the compound in the mixture may be in the salt form (e.g., HCl salt form, Tris salt form, or bis-Tris salt form), calculated as the ratio of the weight of the salt form to the total weight of the mixture.

### III. Formulations

Disclosed are pharmaceutical formulations containing a compound or composition described herein. Generally, the pharmaceutical formulations also contain one or more pharmaceutically acceptable excipients.

The pharmaceutical formulations can be in a form chosen from tablets, capsules, caplets, pills, powders, beads, granules, particles, creams, gels, solutions (such as aqueous solutions, e.g., buffer, saline, and buffered saline), emulsions, suspensions (including nano- and micro-suspensions), sprays, inhalants, nanoparticulate formulations, etc. In some embodiments, the pharmaceutical formulations are oral formulations. In some embodiments, the pharmaceutical formulations are intravenous formulations. In some embodiments, the pharmaceutical formulations are intramuscular formulations. In some embodiments, the pharmaceutical formulations are topical formulations. In some embodiments, the pharmaceutical formulations are intranasal formulations. In some embodiments, the pharmaceutical formulations are inhalation formulations.

In some embodiments, the pharmaceutical formulations are in the form of tablets, caplets, capsules, or pills. In some embodiment, the tablets, caplets, capsules, and pills have an enteric coating to prevent the gastric acids in the stomach from dissolving or degrading the active ingredients. Exemplary enteric coatings are known in the art and described in the sections below.

As used herein, “emulsion” refers to a mixture of non-miscible components homogenously blended together. In some forms, the non-miscible components include a lipophilic component and an aqueous component. For example, an emulsion may be a preparation of one liquid distributed in small globules throughout the body of a second liquid. The dispersed liquid is the discontinuous phase, and the dispersion medium is the continuous phase. When oil or an oleaginous substance is the dispersed liquid and water or an aqueous solution is the continuous phase, it is known as an oil-in-water emulsion, whereas when water or an aqueous solution is the dispersed phase and oil or an oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to degradation or breakdown of a polymeric material into smaller (e.g., non-polymeric) subunits or digestion of the material into smaller subunits.

As used herein, “enteric polymers” refers to polymers that become soluble in the higher pH environment of the lower gastrointestinal tract or slowly erode as they pass through the gastrointestinal tract.

As used herein, “nanoparticulate formulations” generally refers to formulations containing nanoparticles, which are particles having a diameter from about 1 nm to about 1000 nm, from about 10 nm to about 1000 nm, from about 100 nm to about 1000 nm, or from about 250 nm to about 1000 nm. In some embodiments, “nanoparticulate formulations” can also refer to formulations containing microparticles, which are particles having a diameter from about 1 micron to about 100 microns, from about 1 to about 50 microns, from about 1 to about 30 microns, from about 1 micron to about 10 microns. In some embodiments, the nanoparticulate formulation may contain a mixture of nanoparticles, as defined above, and microparticles, as defined above.

As used herein, “surfactant” refers to any agent which preferentially absorbs to an interface between two immiscible phases, such as the interface between water (or aqueous solution) and an organic solvent (or organic solution), between water (or aqueous solution) and air, or between organic solvent (or organic solution) and air. Surfactants generally possess a hydrophilic moiety and a lipophilic moiety.

As used herein, “gel” is a semisolid system containing a dispersion of the active ingredient, i.e., a compound or composition according to the present disclosure, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely dispersed polymer chains that are water-insoluble, where the polymer molecules are in the external or dispersion phase and water (or an aqueous solution) forms the internal or dispersed phase. The polymer chains can be chemically cross-linked (chemical gels) or physically cross-linked (physical gels). Chemical gels possess polymer chains connected through covalent bonds, whereas physical gels have polymer chains linked by non-covalent interactions, such as van der Waals interactions, ionic interactions, hydrogen bonding interactions, and hydrophobic interactions.

As used herein, “beads” refers to beads made with the active ingredient (i.e., a compound or composition according to the present disclosure) and one or more pharmaceutically acceptable excipients. The beads can be produced by applying the active ingredient to an inert support, e.g., inert sugar core coated with the active ingredient. Alternatively, the beads can be produced by creating a “core” comprising both the active ingredient and at least one of the one or more pharmaceutically acceptable excipients. As used herein, “granules” refers to a product made by processing particles of the active ingredient (i.e., a compound or composition according to the present disclosure) that may or may not include one or more pharmaceutical acceptable excipients. Typically, granules do not contain an inert support and are bigger in size compared to the particles used to produce them. Although beads, granules and particles may be formulated to provide immediate release, beads and granules are usually employed to provide delayed release.

As used herein, “enzymatically degradable polymers” refers to polymers that are degraded by bacterial enzymes present in the intestines and/or lower gastrointestinal tract.

### A. Physical Forms and Unit Dosages

Depending upon the administration route, the compounds or compositions described herein may be formulated in a variety of ways. The pharmaceutical formulations can be prepared in various forms, such as tablets, capsules, caplets, pills, granules, powders, nanoparticle formulations, solutions (such as aqueous solutions, e.g., buffer, saline, and buffered saline), suspensions (including nano- and micro-suspensions), emulsions, creams, gels, and the like.

In some embodiments, the pharmaceutical formulations are in a solid dosage form suitable for simple administration of precise dosages. For example, the solid dosage form may be selected from tablets, soft or hard gelatin or non-gelatin capsules, and caplets for oral administration. In some embodiments, the solid dosage form is hard gelatin capsules. Optionally, the solid dosage form is a lyophilized powder that can be readily dissolved and converted to a liquid dosage form for intravenous or intramuscular administration.

In some embodiments, the pharmaceutical formulations are in a liquid dosage form suitable for intravenous or intramuscular administration. Exemplary liquid dosage forms include, but are not limited to, solutions, suspensions, and emulsions. In some embodiments, the pharmaceutical formulations are in the form of a sterile aqueous solution. In some embodiments, the sterile aqueous solution is a sterile normal saline solution. In some embodiments, the sterile aqueous solution is a sterile PBS solution. In some embodiments, the sterile aqueous solution is manufactured by dissolving a lyophilized powder containing the active ingredient (i.e., a compound or composition disclosed herein) in an aqueous medium. For example, the sterile aqueous solution can be prepared by dissolving the lyophilized powder containing the active ingredient in a dose-appropriate volume of sterile water, sterile normal saline, or sterile PBS.

In some embodiments, the pharmaceutical formulations are in a unit dosage form, and may be suitably packaged, for example, in a box, blister, vial, bottle, syringe, sachet, ampoule, or in any other suitable single-dose or multi-dose holder or container, optionally with one or more leaflets containing product information and/or instructions for use.

### B. Pharmaceutically Acceptable Excipients

Exemplary pharmaceutically acceptable excipients include, but are not limited to, diluents, binders, lubricants, disintegrants, pH-modifying or buffering agents, salts (such as NaCl), preservatives, antioxidants, solubility enhancers, wetting or emulsifying agents, plasticizers, colorants (such as pigments and dyes), flavoring or sweetening agents, thickening agents, emollients, humectants, stabilizers, glidants, solvents or dispersion mediums, surfactants, pore formers, and coating or matrix materials.

In some embodiments, the powders described herein, including the lyophilized powders, contain one or more of the following pharmaceutically acceptable excipients: pH-modifying or buffering agents, salts (such as NaCl), and preservatives.

In some embodiments, the tablets, beads, granules, and particles described herein contain one or more of the following pharmaceutically acceptable excipients: coating or matrix materials, diluents, binders, lubricants, disintegrants, pigments, stabilizers, and surfactants. If desired, the tablets, beads, granules, and particles may also contain a minor amount of nontoxic auxiliary substances such as wetting or emulsifying agents, dyes, pH-buffering agents, and preservatives.

Examples of the coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate, or ethyl methacrylate, as well as methacrylic resins that are commercially available under the tradename EUDRAGIT®), enzymatically degradable polymers (such as azo polymers, pectin, chitosan, amylose, and guar gum), zein, shellac, and polysaccharides. In some embodiments, the coating or matrix materials may contain one or more excipients such as plasticizers, colorants, glidants, stabilizers, pore formers, and surfactants.

In some embodiments, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above.

In some embodiments, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some embodiments, the coating or matrix materials are selected such that the pharmaceutical formulations can survive exposure to gastric acid and release the active ingredient in the intestines after oral administration.

Diluents can increase the bulk of a solid dosage formulation so that a practical size is provided for compression of tablets or formation of beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a tablet, bead, granule, or particle remains intact after the formation of the solid dosage formulation. Suitable binders include, but are not limited to, starch, pregelatinized starch, gelatin, sugars (such as sucrose, glucose, dextrose, lactose, and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, and synthetic polymers (such as acrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate disintegration or “breakup” of a solid dosage formulation after administration. Suitable disintegrants include, but are not limited to, starch, sodium starch glycolate, sodium carboxymethyl starch, sodium carboxymethylcellulose, hydroxypropyl cellulose, pregelatinized starch, clays, cellulose, gums, and cross-linked polymers, such as cross-linked polyvinylpyrrolidone (e.g., POLYPLASDONE® XL).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active ingredient in the pharmaceutical formulations or stabilize particles in a dispersion. For example, when the decomposition reactions involve an oxidation reaction of the active ingredient in the pharmaceutical formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidants include, but are not limited to, talc, magnesium stearate, and glycerol monostearates.

Preservatives can inhibit the deterioration and/or decomposition of a pharmaceutical formulation. Deterioration or decomposition can be brought about by one or more of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, propionate salts (e.g., sodium propionate), chlorobutanol, benzyl alcohol, and combinations thereof.

Surfactants may be anionic, cationic, amphoteric, or nonionic surface-active agents. Exemplary anionic surfactants include, but are not limited to, those containing a carboxylate, sulfonate, or sulfate ion. Examples of anionic surfactants include sodium, potassium, and ammonium salts of long-chain (e.g., 13-21) alkyl sulfonates (such as sodium lauryl sulfate), alkylaryl sulfonates (such as sodium dodecylbenzene sulfonate), and dialkyl sulfosuccinates (such as sodium bis-(2-ethylthioxyl)-sulfosuccinate). Examples of cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, poloxamers (such as poloxamer 401), stearoyl monoisopropanolamide, and polyoxyethylene hydrogenated tallow amide. Examples of amphoteric surfactants include, but are not limited to, sodium N-dodecyl-β-alanine, sodium N-lauryl-β-iminodipropionate, myristoamphoacetate, lauryl betaine, and lauryl sulfobetaine.

Pharmaceutical formulations in the liquid dosage forms typically contain a solvent or dispersion medium such as water, aqueous solution (e.g., buffer, saline, buffered saline), ethanol, polyol (such as glycerol, propylene glycol, and polyethylene glycol), oil (such as vegetable oil, e.g., peanut oil, corn oil, sesame oil), and combinations thereof. In some embodiments, the pharmaceutical formulations in the liquid dosage forms are aqueous formulations. Suitable solvents or dispersion mediums for aqueous formulations include, but are not limited to, water, buffers (such as acidic buffers), salines (such as normal saline), buffered salines (such as PBS), and Ringer's solution.

In some embodiments, the pharmaceutical formulations contain β-cyclodextrin or derivatives thereof. For example, the pharmaceutical formulations contain sulfobutyl ether β-cyclodextrin. For example, the pharmaceutical formulations contain hydroxypropyl-β-cyclodextrin. Such pharmaceutical formulations can be in a solid dosage form or a liquid dosage form.

### C. Pharmaceutical Acceptable Carriers

In some embodiments, the pharmaceutical formulations are prepared using a pharmaceutically acceptable carrier, which encapsulates, embeds, entraps, dissolves, disperses, absorbs, and/or binds to a compound or composition disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound or composition in performing its function. The pharmaceutically acceptable carrier can be formed of biodegradable materials, non-biodegradable materials, or combinations thereof. One or more of the pharmaceutical acceptable excipients described above may be present in the pharmaceutical acceptable carrier.

In some embodiments, the pharmaceutical acceptable carrier is a controlled-release carrier, such as delayed-release carriers, sustained-release (extended-release) carriers, and pulsatile-release carriers.

In some embodiments, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to: nanoparticles, microparticles, and combinations thereof, liposomes; hydrogels; polymer matrices; and solvent systems.

In some embodiments, the pharmaceutical acceptable carrier is nanoparticles, microparticles, or a combination thereof. In some embodiments, the compound or composition is embedded in the matrix formed by the materials of the nanoparticles, microparticles, or combination thereof.

The nanoparticles, microparticles, or combination thereof can be biodegradable, and optionally are capable of biodegrading at a controlled rate for delivery of the compound or composition. The nanoparticles, microparticles, or combination thereof can be made of a variety of materials. Both inorganic and organic materials can be used. Both polymeric and non-polymeric materials can be used.

For example, the nanoparticles, microparticles, or combination thereof are formed of one or more biocompatible polymers. In some forms, the biocompatible polymers are biodegradable. In some forms, the biocompatible polymers are non-biodegradable. In some forms, the nanoparticles, microparticles, or combination thereof are formed of a mixture of biodegradable and non-biodegradable polymers. The polymers used to form the nanoparticles, microparticles, or combination thereof may be tailored to optimize different characteristics of the nanoparticles, microparticles, or combination thereof, including: (i) interactions between the active ingredient and the polymer to provide stabilization of the active ingredient and retention of activity upon delivery; (ii) rate of polymer degradation and, thereby, rate of release; (iii) surface characteristics and targeting capabilities; and (iv) particle porosity.

Exemplary polymers include, but are not limited to, polymers prepared from lactones (such as poly(caprolactone) (PCL)), polyhydroxy acids and copolymers thereof (such as poly(lactic acid) (PLA), poly(glycolic acid) (PGA), and poly(lactic acid-co-glycolic acid) (PLGA)), polyalkyl cyanoacralate, polyurethanes, polyamino acids (such as poly-L-lysine (PLL), poly(valeric acid), and poly-L-glutamic acid), hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters (such as poly(vinyl acetate)), polyvinyl halides (such as poly(vinyl chloride) (PVC)), polyvinylpyrrolidone, polysiloxanes, polystyrene (PS), celluloses and derivatized celluloses (such as alkyl celluloses, hydroxyalkyl celluloses, cellulose ethers, cellulose esters, nitro celluloses, hydroxypropylcellulose, and carboxymethylcellulose), polymers of acrylic acids (such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate)), polydioxanone, polyhydroxyalkanoates, polypropylene fumarate, polyoxymethylene, poloxamers, poly(butyric acid), polyphosphazenes, polysaccharides, polypeptides, and blends thereof.

In some embodiments, the one or more biocompatible polymers forming the nanoparticles, microparticles, or combination thereof include an FDA-approved biodegradable polymer such as polyhydroxy acids (e.g., PLA, PGA, and PLGA), polyanhydrides, and polyhydroxyalkanoate (e.g., poly(3-butyrate) and poly(4-butyrate)).

Materials other than polymers may be used to form the nanoparticles, microparticles, or combination thereof. Suitable materials include surfactants. The use of surfactants in the nanoparticles, microparticles, or combination thereof may improve surface properties by, for example, reducing particle-particle interactions, and render the surface of the particles less adhesive. Both naturally occurring surfactants and synthetic surfactants can be incorporated into the nanoparticles, microparticles, or combination thereof. Exemplary surfactants include, but are not limited to, phosphoglycerides such as phosphatidylcholines (e.g., L-α-phosphatidylcholine dipalmitoyl), diphosphatidyl glycerol, hexadecanol, fatty alcohols, polyoxyethylene-9-lauryl ether, fatty acids such as palmitic acid and oleic acid, sorbitan trioleate, glycocholate, surfactin, poloxomers, sorbitan fatty acid esters such as sorbitan trioleate, tyloxapol, and phospholipids.

The nanoparticles, microparticles, or combination thereof may contain a plurality of layers. The layers can have similar or different release kinetic profiles for the active ingredient. For example, the nanoparticles, microparticles, or combination thereof can have a controlled-release core surrounded by one or more additional layers. The one or more additional layers can include an instant-release layer, preferably on the surface of the nanoparticles, microparticles, or combination thereof. The instant-release layer can provide a bolus of the active ingredient shortly after administration.

The composition and structure of the nanoparticles, microparticles, or combination thereof can be selected such that the nanoparticles, microparticles, or combination thereof are pH-sensitive or pH-responsive. In some embodiments, the nanoparticles, microparticles, or combination thereof are formed of one or more pH-sensitive or pH-responsive polymers such as the enteric polymers commercially available under the tradename EUDRAGIT®, as described above. Depending on the particle materials, the decomposition/degradation or structural change of the nanoparticles, microparticles, or combination thereof may occur at different locations of the gastrointestinal tract. In some embodiments, the particle materials are selected such that the nanoparticles, microparticles, or combination thereof can survive exposure to gastric acid and release the active ingredient in the intestines after oral administration.

### D. Controlled Release

In some embodiments, the pharmaceutical formulations can be controlled-release formulations. Examples of controlled-release formulations include extended-release formulations, delayed-release formulations, and pulsatile-release formulations.

### 1. Extended Release

In some embodiments, the extended-release formulations are prepared as diffusion or osmotic systems, for example, as described in “Remington—The science and practice of pharmacy” (23rd Ed., Elsevier, 2021).

A diffusion system is typically in the form of a matrix, generally prepared by combining the active ingredient with a slowly dissolving, pharmaceutically acceptable carrier, optionally in a tablet form. Suitable materials used in the preparation of the matrix include plastics, hydrophilic polymers, and fatty compounds. Suitable plastics include, but are not limited to, acrylic polymer, methyl acrylate-methyl methacrylate copolymer, polyvinyl chloride, and polyethylene. Suitable hydrophilic polymers include, but are not limited to, cellulosic polymers such as methyl ethyl cellulose, hydroxyalkylcelluloses (such as hydroxypropylcellulose and hydroxypropylmethylcellulose), sodium carboxymethylcellulose, CARBOPOL® 934, polyethylene oxides, and combinations thereof. Suitable fatty compounds include, but are not limited to, various waxes such as carnauba wax and glyceryl tristearate, wax-type substances such as hydrogenated castor oil and hydrogenated vegetable oil, and combinations thereof.

In some embodiments, the plastic is a pharmaceutically acceptable acrylic polymer. In some embodiments, the pharmaceutically acceptable acrylic polymer is chosen from acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid), polymethacrylate, polyacrylamide, poly(methacrylic acid anhydride), and glycidyl methacrylate copolymers.

In some embodiments, the pharmaceutically acceptable acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the pharmaceutically acceptable acrylic polymer is an acrylic resin lacquer such as those commercially available under the tradename EUDRAGIT®. In some embodiments, the pharmaceutically acceptable acrylic polymer contains a mixture of two acrylic resin lacquers, EUDRAGIT® RL (such as EUDRAGIT® RL30D) and EUDRAGIT® RS (such as EUDRAGIT® RS30D). EUDRAGIT® RL30D and EUDRAGIT® RS30D are copolymers of acrylic and methacrylic acid esters with a low content of quaternary ammonium groups, the molar ratio of ammonium groups to the remaining neutral methacrylic esters being 1:20 in EUDRAGIT® RL30D and 1:40 in EUDRAGIT® RS30D. The code designations RL (high permeability) and RS (low permeability) refer to the permeability properties of these polymers. EUDRAGIT® RL/RS mixtures are insoluble in water and in digestive fluids. However, multi-particulate systems formed to include the same are swellable and permeable in aqueous solutions and digestive fluids. The EUDRAGIT® RL/RS mixtures may be prepared in any desired ratio in order to ultimately obtain a sustained-release formulation having a desirable release profile. Suitable sustained-release, multi-particulate systems may be obtained, for instance, from 90% EUDRAGIT® RL+10% EUDRAGIT® RS, to 50% EUDRAGIT® RL+50% EUDRAGIT® RS, and to 10% EUDRAGIT® RL+90% EUDRAGIT® RS. In some embodiments, the pharmaceutically acceptable acrylic polymer can also be or include other acrylic resin lacquers, such as EUDRAGIT® 5-100, EUDRAGIT® L-100, and mixtures thereof.

Matrices with different release mechanisms or profiles can be combined in a final dosage form containing single or multiple units. Examples of multiple units include, but are not limited to, multilayer tablets and capsules containing beads, granules, and/or particles of the active ingredient. An immediate release portion can be added to the extended-release system by means of either applying an immediate release layer on top of the extended-release core using a coating or compression process or in a multiple unit system such as a capsule containing both extended- and immediate-release beads.

Extended-release tablets containing one or more of the hydrophilic polymers can be prepared by techniques commonly known in the art such as direct compression, wet granulation, and dry granulation.

Extended-release tablets containing one or more of the fatty compounds can be prepared using methods known in the art such as direct blend methods, congealing methods, and aqueous dispersion methods. In the congealing methods, the active ingredient is mixed with the fatty compound(s) and congealed.

Alternatively, the extended-release formulations can be prepared using osmotic systems or by applying a semi-permeable coating to a solid dosage form. In the latter case, the desired release profile can be achieved by combining low permeable and high permeable coating materials in suitable proportions.

### 2. Delayed Release

Delayed-release formulations can be prepared by coating a solid dosage form with a coating. In some embodiments, the coating is insoluble and impermeable in the acidic environment of the stomach and becomes soluble or permeable in the less acidic environment of the intestines and/or the lower GI tract. In some embodiments, the solid dosage form is a tablet for incorporation into a capsule, a tablet for use as an inner core in a “coated-core” dosage form, or a plurality of beads, granules, and/or particles containing the active ingredient, for incorporation into either a tablet or capsule.

Suitable coating materials may be bioerodible polymers, gradually hydrolysable polymers, gradually water-dissolvable polymers, and enzymatically degradable polymers. In some embodiments, the coating material is or contains enteric polymers. Combinations of different coating materials may also be used. Multilayer coatings using different coating materials may also be applied.

The coating may also contain one or more additives, such as plasticizers as described above (optionally representing about 10 wt % to 50 wt % relative to the dry weight of the coating), colorants as described above, stabilizers as described above, glidants as described above, etc.

### 3. Pulsatile Release

Pulsatile-release formulations release a plurality of doses of the active ingredient intermittently. Generally, upon administration, such as oral administration, of the pulsatile-release formulations, release of the initial dose is substantially immediate, e.g., the first release “pulse” occurs within about three hours, two hours, or one hour of administration. This initial pulse may be followed by a first time-interval (lag time) during which very little or no active ingredient is released from the formulations, after which a second dose may be released. Similarly, a second lag time (nearly release-free interval) between the second and third release pulses may be designed. The duration of the lag times will vary depending on the formulation design, especially on the length of the dosing interval, e.g., a twice daily dosing profile, a three-time daily dosing profile, etc.

For pulsatile-release formulations providing a twice daily dosage profile, they deliver two release pulses of the active ingredient. In some embodiments, the one nearly release-free interval between the first and second release pulses may have a duration of between 3 hours and 14 hours.

For pulsatile-release formulations providing a three daily dosage profile, they deliver three release pulses of the active ingredient. In some embodiments, the two nearly release-free interval between two adjacent pulses may have a duration of between 2 hours and 8 hours.

In some embodiments, the pulsatile-release formulations contain a plurality of pharmaceutically acceptable carriers with different release kinetics.

In some embodiments, the pulsatile-release formulations contain a pharmaceutically acceptable carrier with a plurality of layers loaded with the active ingredient. In some embodiments, the layers may have different release kinetics. In some embodiments, the layers may be separated by a delayed-release coating. For example, the pulsatile-release formulations may have a first layer loaded with the active ingredient on the surface for the first release pulse and a second layer, e.g., a core loaded with the active ingredient, for the second release pulse; the second layer may be surrounded by a delayed-release coating, which creates a lag time between the two release pulses.

In some embodiments, the pulsatile-release profile is achieved with formulations that are closed and optionally sealed capsules housing at least two “dosage units” wherein each dosage unit within the capsules provides a different release profile. In some embodiments, at least one of the dosage units is a delayed-release dosage unit. Control of the delayed-release dosage unit(s) may be accomplished by a controlled-release polymer coating on the dosage unit(s) or by incorporation of the active ingredient in a controlled-release polymer matrix. In some embodiments, each dosage unit may comprise a compressed or molded tablet, wherein each tablet within the capsule provides a different release profile.

### E. Exemplary Formulations for Different Routes of Administration

A subject suffering from an inflammatory condition, disorder, or disease or a condition, disorder, or disease associated increased mature mast cells as described herein, can be treated by systemic, local, or inhalation administration of a pharmaceutical formulation containing a compound or composition described herein. Exemplary administration routes include, but are not limited to, oral, intramuscular, intravenous, subcutaneous, topical, transdermal, trans- or sub-mucosal, intranasal, and inhalation administrations.

In some embodiments, the pharmaceutical formulation is formulated for oral administration. In some embodiments, the pharmaceutical formulation is formulated for intravenous, intramuscular, or subcutaneous administration. In some embodiments, the pharmaceutical formulation is formulated for inhalation or intranasal administration. In some embodiments, the pharmaceutical formulation is formulated for topical or transdermal administration.

In some embodiments, the pharmaceutical formulation is an oral pharmaceutical formulation. In some embodiments, the active ingredient may be incorporated with one or more pharmaceutically acceptable excipients as described above and used in the form of tablets, pills, caplets, or capsules. For example, the corresponding oral pharmaceutical formulation may contain one or more of the following pharmaceutically acceptable excipients or those of a similar nature: a binder as described above, a disintegrant as described above, a lubricant as described above, a glidant as described above, a sweetening agent (such as sucrose and saccharin), and a flavoring agent (such as methyl salicylate and fruit flavorings). In some embodiments, when the oral pharmaceutical formulation is in the form of capsules, it may contain, in addition to the material(s) listed above, a liquid carrier (such as a fatty oil). In some embodiments, when the oral pharmaceutical formulation is in the form of capsules, each capsule may contain a plurality of beads, granules, and/or particles of the active ingredient. In some embodiments, the oral pharmaceutical formulation may contain one or more other materials which modify the physical form or one or more pharmaceutical properties of the dosage unit, for example, coatings of polysaccharides, shellac, or enteric polymers as described in previous sections.

In some embodiments, the oral pharmaceutical formulation can be in the form of an elixir, suspension, syrup, wafer, chewing gum or the like. A syrup may contain, in addition to the active ingredient, one or more sweetening agents (such as sucrose and saccharine), one or more flavoring agents, one or more preservatives, and/or one or more dyes or colorings.

In some embodiments, the pharmaceutical formulation is an intravenous, intramuscular, or subcutaneous pharmaceutical formulation. In some embodiments, the intravenous, intramuscular, or subcutaneous pharmaceutical formulation can be enclosed in an ampoule, syringe, or a single or multiple dose vial made of glass or plastic. In some embodiments, the intravenous, intramuscular, or subcutaneous pharmaceutical formulation contains a liquid pharmaceutically acceptable carrier for the active ingredient. Suitable liquid pharmaceutically acceptable carriers include, but are not limited to, water, buffer, saline, buffered saline (such as PBS), and combinations thereof.

In some embodiments, the pharmaceutical formulation is a topical pharmaceutical formulation. Suitable forms of the topical pharmaceutical formulation include lotions, suspensions, ointments, creams, gels, tinctures, sprays, powders, pastes, slow-release transdermal patches, and suppositories for application to rectal, vaginal, nasal, or oral mucosa. In some embodiments, thickening agents, emollients (such as mineral oil, lanolin and its derivatives, and squalene), humectants (such as sorbitol), and/or stabilizers can be used to prepare the topical pharmaceutical formulations. Examples of thickening agents include petrolatum, beeswax, xanthan gum, and polyethylene.

In some embodiments, the pharmaceutical formulation is an intranasal pharmaceutical formulation. In some embodiments, the intranasal pharmaceutical formulation is in the form of an aqueous suspension, which can be optionally placed in a pump spray bottle. Other than water, the aqueous suspension may contain one or more pharmaceutically acceptable excipients, such as suspending agents (e.g., microcrystalline cellulose, sodium carboxymethylcellulose, hydroxypropyl-methyl cellulose), humectants (e.g., glycerol, propylene glycol), acids, bases, and/or pH-buffering agents for adjusting the pH (e.g., citric acid, sodium citrate, phosphoric acid, sodium phosphate, and combinations thereof), surfactants (e.g., polysorbate 80), and preservatives (e.g., benzalkonium chloride, phenylethyl alcohol, potassium sorbate).

In some embodiments, the pharmaceutical formulation is an inhalation pharmaceutical formulation. In some embodiments, the inhalation pharmaceutical formulation may be in the form of an aerosol suspension, a dry powder, or a liquid suspension. The inhalation pharmaceutical formulation may be prepared for delivery as a nasal spray or an inhaler, such as a metered dose inhaler (MDI). In some embodiments, MDIs can deliver aerosolized particles suspended in chlorofluorocarbon propellants such as CFC-11 and CFC-12, or non-chlorofluorocarbons or alternate propellants such as fluorocarbons (e.g., HFC-134A, H1FC-227), with or without surfactants or suitable bridging agents. Dry-powder inhalers can also be used, either breath activated or delivered by pressure.

In some embodiments, the active ingredient is prepared with a pharmaceutically acceptable carrier that will protect it against rapid degradation or elimination from the body of the subject after administration, such as the controlled-release formulations described in previous sections.

### IV. Methods of Using

The utility of the compounds, compositions, and pharmaceutical formulations of this disclosure may be administered to a subject to inhibit c-kit. In this context, disclosed are methods of treating or preventing an inflammatory condition, disorder, or disease or a condition, disorder, or disease associated with an increased number of mature mast cells in a subject in need thereof. The methods include administering an effective amount of a compound, composition, or pharmaceutical formulation disclosed herein to the subject.

The compound, composition, or pharmaceutical formulation can be administered in a variety of manners, depending on whether systemic, local, or inhalation administration is desired. Exemplary administration routes include, but are not limited to, oral, intramuscular, intravenous, subcutaneous, topical, transdermal, trans- or sub-mucosal, intranasal, and inhalation administrations.

Optionally, the compound, composition, or pharmaceutical formulation is administered in a systemic manner, such as enteral administration (e.g., oral administration) and parenteral administration (e.g., injection, infusion, and implantation). Exemplary systemic administration routes include oral administration, intravenous administration such as intravenous injection or infusion, intramuscular administration such as intramuscular injection, subcutaneous administration such as subcutaneous injection, intranasal administration, topical administration, and transdermal administrations.

Optionally, the compound, composition, or pharmaceutical formulation is administered locally to a specific bodily location of the subject. Exemplary local administration routes include intranasal administration, trans- or sub-mucosal administration, and topical administration.

Optionally, the compound, composition, or pharmaceutical formulation is administered by inhalation, optionally using a nasal spray or an inhaler.

In some embodiments, the compound, composition, or pharmaceutical formulation is administered orally. In some embodiments, the compound, composition, or pharmaceutical formulation is administered intravenously. In some embodiments, the compound, composition, or pharmaceutical formulation is administered intramuscularly. In some embodiments, the compound, composition, or pharmaceutical formulation is administered topically. In some embodiments, the compound, composition, or pharmaceutical formulation is administered intranasally. In some embodiments, the compound, composition, or pharmaceutical formulation is administered by inhalation.

In some embodiments, the subject is a human. In some embodiments, the subject is an adult patient. In some embodiments, the subject is a pediatric patient. In some embodiments, the subject is a non-human animal, such as domestic pets, livestock and farm animals, and zoo animals. In some embodiments, the non-human animal may be a non-human primate.

### A. Indications

The condition, disorder, or disease may be characterized by an inflammatory response related to c-kit or to an increased number of mature mast cells. For example, the condition, disorder, or disease may be caused, induced, or mediated, at least partially, by c-kit. In some embodiments, the condition, disorder, or disease is related to c-kit upregulation or hyperactivity. For example, the condition, disorder, or disease may be caused, induced, or mediated, at least partially, by c-kit upregulation or hyperactivity. As used herein, “upregulation” refers to an increase in c-kit mRNA or protein, compared to the corresponding basal level. The upregulation may be caused by a stimulus, such as an antigen or allergen. As used herein, “hyperactivity” refers to an activity higher than the normal activity in a defined population. The hyperactivity may be caused by a genetic mutation (such as gain-of-function mutation) or other genetic abnormality or polymorphism of c-kit. Exemplary genetic mutations of c-kit may include those in Exon 9, Exon 11, Exon 13, Exon 14, Exon 17, and Exon 18. In some embodiments, the hyperactivity is caused by D816V, D816H, or D816Y mutation in Exon 17.

Further, the inflammatory response of the condition, disorder, or disease may be related to mast cells, which express c-kit on their surface. For example, the condition, disorder, or disease may be caused, induced, or mediated, at least partially, by mast cells. In some embodiments, the condition, disorder, or disease is related to mast cell activation, such as an overabundance of mature mast cells. For example, the condition, disorder, or disease may be caused, induced, or mediated, at least partially, by mast cell activation, such as an overabundance of mature mast cells. As used herein, “mast cell activation” refers to the transition of immature (dormant) mast cells (mast cell progenitors) to mature mast cells. As shown in the Examples, mast cell activation can be assessed by measuring the ratio between immature mast cells to mature mast cells in a tissue, organ, or system of the subject, such as the blood or spleen. Alternatively, mast cell activation can be assessed by measuring the population of mature mast cells. In some embodiments, mast cell activation may be related to c-kit upregulation or hyperactivity, as described above. In some embodiments, mast cell activation may be related to a genetic mutation, genetic abnormality, or genetic polymorphism not associated with c-kit.

Optionally, the inflammatory response is an allergic reaction or a reaction to a pathogen, including anaphylactic episodes of the allergic reaction. In some embodiments, the inflammatory response is related to c-kit upregulation or hyperactivity, as described above. In some embodiments, the inflammatory response is related to mast cell activation, such as an overabundance of mature mast cells, as described above. In some embodiments, the compound, composition, or pharmaceutical formulation is used to reduce the severity and/or intensity of the inflammatory response. In some embodiments, the compound, composition, or pharmaceutical formulation is used to reduce the frequency of episodes of the inflammatory response. In some embodiments, the compound, composition, or pharmaceutical formulation is used to prevent an anaphylactic episode of an allergic reaction.

In some embodiments, the inflammation occurs in a tissue, organ, or system of the subject, selected from skin, connective tissues (e.g., cartilage or bone), mucosal tissues, joints, organs (e.g., gastrointestinal organs, lungs, spleen, pancreas, liver), cardiovascular system, lymphatic system, skeletal system, respiratory system, and digestive system.

Exemplary conditions relevant to this disclosure include, but are not limited to, mast cell diseases (e.g., mastocytosis, mast cell activation syndrome (MCAS), hereditary alpha tryptasemia (HαT)), urticaria, Lyme disease, mast cell leukemia, lung diseases (e.g., chronic obstructive pulmonary disease (COPD), idiopathic pulmonary fibrosis), long COVID, asthma, allergy, inflammatory bowel disease, arthritis, and gout. In some embodiments, the inflammatory condition to be treated using the compounds, compositions, or pharmaceutical composition described herein is a mast cell disease. According to the Mast Cell Disease Society, mast cell disease is a term used by mast cell specialists for all mast cell-related diseases. This term covers various form of mastocytosis, MCAS, and HαT. Mastocytosis refers to a group of disorders characterized by excessive mast cell accumulation in one tissue or in multiple tissues. Mastocytosis may be divided into two groups of disorders: (1) cutaneous mastocytosis (CM), forms that are limited to the skin, and (2) systemic mastocytosis (SM), forms in which mast cells infiltrate extracutaneous organs, with or without skin involvement. SM is further subdivided into five forms: indolent (ISM), smoldering (SSM), aggressive (ASM), SM with associated hemotologic non-mast cell lineage disease (SM-AHNMD), and mast cell leukemia (MCL).

In some embodiments, the inflammatory condition is systemic mastocytosis. Diagnosis of systemic mastocytosis is based in part on histological and cytological studies of bone marrow showing infiltration by mast cells of frequently atypical morphology, which frequently abnormally express non-mast cell markers (CD25 and/or CD2). Diagnosis of systemic mastocytosis is confirmed when bone marrow mast cell infiltration occurs in the context of one of the following: (1) abnormal mast cell morphology (spindle-shaped cells); (2) elevated level of serum tryptase above 20 ng/mL; or (3) the presence of the activating c-kit D816V mutation. Activating mutations at the D816 position of c-kit are found in the vast majority of mastocytosis cases, with the most common mutations being D816V, D816H, and D816Y The D816V mutation is found in the activation loop of the kinase domain and leads to constitutive activation of c-kit.

In some embodiments, the inflammatory condition is MCAS. MCAS is a condition in which the subject experiences repeated anaphylactic episodes with symptoms such as hives, swelling, low blood pressure, breathing difficulty, and severe diarrhea. High levels of mast cell mediators are released during those episodes. In MCAS, mast cells mistakenly release too many immune factors (e.g., cytokines), resulting in multi-organ symptoms.

According to the Mast Cell Disease Society, variants of MCAS include primary MCSA, secondary MCAS, and idiopathic MCAS. Primary MCAS results from a clonal population of mast cells, where a genetic alteration in the cells exists, and may be due to mastocytosis or to monoclonal Mast Cell Activation Syndrome (MMAS). Primary MCAS with mastocytosis can be diagnosed if the subject meets the criteria for MCAS and the WHO criteria for mastocytosis. MMAS is a distinct disease characterized by the presence of abnormal mast cells and fulfillment of criteria for MCAS, but where sufficient criteria for a diagnosis of mastocytosis are not identified. Secondary MCAS is diagnosed when mast cell activation occurs as an indirect result of another disease or condition. In addition to the widespread example of IgE-dependent allergy as a cause of secondary MCAS, other diseases that can cause secondary MCAS. Idiopathic MCAS is proposed as a final diagnosis after proposed MCAS criteria have been fulfilled and a thorough evaluation has excluded the possibility of another known underlying cause for this activation.

In some embodiments, the inflammatory condition is Lyme disease. For example, the Lyme disease may be post-treatment Lyme disease or chronic Lyme disease. Upon infection by a Lyme disease-inducing bacterium such as Borrelia burgdorferi, mast cells are activated as part of a normal immune response. Sometimes, even after the Lyme disease-inducing bacterium has been eliminated, the Lyme disease symptoms prevail, as a result of chronically activated mast cells in tissues and organs. This chronic post-infectious mast cell activation causes a wide spectrum of devastating symptoms similar to the symptoms of MCAS patients. Further, the intensive use of antibiotics to treat Lyme disease in many subjects leads to a disturbed intestinal permeability and an increased uptake of antigens that stimulate mast cell activation and amplify the systemic chronic activation and further accumulation of mast cells. See Infect. Immun., 1999, 67(3):1107-1115 and BMC Public Health, 2019, 19:352.

In some embodiments, the inflammatory condition is COPD. The pathogenesis of COPD is based on the innate and adaptive inflammatory immune response to the inhalation of toxic particles and gases. Although tobacco smoking is the primary cause of COPD, many other environmental and occupational exposures can cause or contribute to COPD. The inflamed airways of COPD patients contain several inflammatory cells including neutrophils, macrophages, T lymphocytes, and dendritic cells. Mast cell activation is involved in the pathogenesis of COPD, as documented in Eur. J. Pharmacol., 2016, 778:125-138 and Pulm. Pharmacol. Ther., 2011, 24(4):367-72.

In some embodiments, the inflammatory condition is long COVID. Mast cell activation is involved in the pathogenesis of long COVID, as documented in Nature Reviews Microbiology, 2023, 21:133-146 and Br. J. Hosp. Med. (Lond), 2022, 83(7):1-10.

In some embodiments, the inflammatory condition is not a neurodegenerative disease, neurodevelopmental disorder, myodegenerative disease, prion disease, or lysosomal storage disease.

### B. Dosing and Administration

The compound, composition, or pharmaceutical formulation may be administered to a subject for a sufficient time period to alleviate one or more undesired symptoms and/or one or more clinical signs associated with the condition, disorder, or disease being treated.

In some embodiments, the compound, composition, or pharmaceutical formulation is administered once or multiple times daily, including once or twice daily. Each administration can be a single oral dosage, a single intravenous dosage, a single topical dosage, or a single inhalation dosage.

In some embodiments, the compound of this disclosure is administered with a second therapeutic agent. The second therapeutic agent may be selected from non-steroidal anti-inflammatory agents, steroids, bronchodilators, and biologic agents. In some embodiments, the second therapeutic agent is another c-kit inhibitor, such as imatinib, nilotinib, sunitinib, bosutinib, regorafenib, and avapritinib. The second therapeutic agent may be administered before, concurrent with, or after the administration of the compound of this disclosure.

### V. Additional Definitions

As used herein, the singular forms “a,” “an,” and “the” include plural referents unless the context clearly dictates otherwise.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

A carbon range (e.g., C₁₋₁₀) is intended to disclose individually every possible carbon value and/or sub-range encompassed within. For example, a carbon range of C₁₋₁₀ discloses C₁, C₂, C₃, C₄, C₅, C₆, C₇, C₈, C₉, and C₁₀, as well as sub-ranges encompassed therein, such as C₂₋₉, C₃₋₈, C₁₋₅, etc.

As used herein, the term “subject” refers to an animal, including human and non-human animals. Human subjects may include pediatric patients and adult patients. Non-human animals may include domestic pets, livestock and farm animals, and zoo animals. In some cases, the non-human animals may be non-human primates.

As used herein, the terms “prevent” and “preventing” include the prevention of the occurrence, onset, spread, and/or recurrence. It is not intended that the present disclosure is limited to complete prevention. For example, prevention is considered as achieved when the occurrence is delayed, the severity of the onset is reduced, or both. In the context of an inflammatory condition, disease, or disorder, prevention includes delaying, eliminating, or reducing the intensity of a flare in the condition, disease, or disorder.

As used herein, the terms “treat” and “treating” include medical management of a condition, disorder, or disease of a subject as would be understood by a person of ordinary skill in the art (see, for example, Stedman's Medical Dictionary). In general, treatment is not limited to cases where the subject is cured and the condition, disorder, or disease is eradicated. Rather, treatment also contemplates cases where a treatment regimen containing one of the compounds, compositions, or pharmaceutical formulations of the present disclosure provides an improved clinical outcome. The improved clinical outcome may include one or more of the following: abatement, lessening, and/or alleviation of one or more symptoms that result from or are associated with the condition, disorder, or disease to be treated; decreased occurrence of one or more symptoms; improved quality of life; diminishment of the extent of the condition, disorder, or disease; reaching or establishing a stabilized state (i.e., not worsening) of the condition, disorder, or disease; delay or slowing of the progression of the condition, disorder, or disease; amelioration or palliation of the state of the condition, disorder, or disease; partial or total remission; and improvement in survival (whether increase in the overall survival rate or prolonging of survival when compared to expected survival if the subject were not receiving the treatment). For example, the disclosure encompasses treatment that reduces one or more symptoms of and/or cognitive deficit associated with or caused by a brain injury.

The terms “derivative” and “derivatives” refer to chemical compounds/groups/moieties with a structure similar to that of a parent compound/group/moiety but different from it in respect to one or more components, functional groups, atoms, etc. Optionally, the derivatives retain certain functional attributes of the parent compound/group/moiety. Optionally, the derivatives can be formed from the parent compound/group/moiety by chemical reaction(s). The differences between the derivatives and the parent compound/group/moiety can include, but are not limited to, replacement of one or more functional groups with one or more different functional groups or introducing or removing one or more substituents of hydrogen atoms.

The term “alkyl” refers to univalent groups derived from alkanes (i.e., acyclic saturated hydrocarbons) by removal of a hydrogen atom from any carbon atom. Alkyl groups can be linear or branched. Suitable alkyl groups can have one to 20 carbon atoms, i.e., C₁₋₂₀ alkyl. If the alkyl is branched, it is understood that at least three carbon atoms are present.

The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner, a fused manner, or a combination thereof. Accordingly, in polycyclic aryl groups, the rings can be attached together in a pendant manner, a fused manner, or a combination thereof. Suitable aryl groups can have six to 20 carbon atoms, i.e., C₆₋₂₀ aryl. The number of “members” of an aryl group refers to the total number of carbon atoms in the ring(s) of the aryl group.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). Heteroarenes can be monocyclic or polycyclic. In polycyclic heteroarenes, the rings can be attached together in a pendant manner, a fused manner, or a combination thereof. Accordingly, in polycyclic heteroaryl groups, the rings can be attached together in a pendant manner, a fused manner, or a combination thereof. Suitable heteroaryl groups can have one to 20 carbon atoms, i.e., C₁₋₂₀ heteroaryl. The number of “members” of a heteroaryl group refers to the total number of carbon atom(s) and heteroatom(s) in the ring(s) of the heteroaryl group.

The term “heterocyclyl” refers to mono- or polycyclic, univalent ring systems containing at least one carbon atom and one or more heteroatoms independently selected from elements like N, O, and S, as ring atoms. Optionally, the N and/or S heteroatom(s) may be oxidized, and the N heteroatom(s) may be quaternized. The mono- and polycyclic ring systems may be aromatic, non-aromatic, or a mixture of aromatic and non-aromatic rings. Heterocyclyls may include heteroaryls. In polycyclic heterocyclyl groups, the rings can be attached together in a pendant manner (i.e., two rings are connected by a single bond), a spiro manner (i.e., two rings are connected through a defining single common atom), a fused manner (i.e., two rings share two adjacent atoms; in other words, two rings share one covalent bond), a bridged manner (i.e., two rings share three or more atoms, separating the two bridgehead atoms by a bridge containing at least one atom), or a combination thereof. Suitable heterocyclyl groups can have one to 20 carbon atoms, i.e., C₁₋₂₀ heterocyclyl. The number of “members” of a heterocyclyl group refers to the total number of carbon atom(s) and heteroatom(s) in the ring(s) of the heterocyclyl group.

As used herein, the terms “halogen” and “halo” refer to fluorine, chlorine, bromine, and iodine.

The term “substituted,” as used herein, means that the chemical group or moiety contains one or more substituents replacing the hydrogen atom(s) in the original chemical group or moiety. It is understood that any substitution is in accordance with a permitted valence of the substituted atom and the substituent and that the substitution results in a stable compound, e.g., a compound that does not spontaneously undergo transformation such as by rearrangement, cyclization, elimination, etc., under room temperature.

As used herein, the term “stereoisomer” refers to compounds made up of the same atoms having the same bond order but having different three-dimensional arrangements of atoms which are not interchangeable. As used herein, the term “enantiomer” refers to a pair of stereoisomers that are non-superimposable mirror images of one another. As used herein, the term “diastereomer” refers to two stereoisomers that are not mirror images but also not superimposable. The terms “racemate” and “racemic mixture” refer to a mixture of enantiomers. The term “chiral center” refers to a carbon atom to which four different groups are attached. Choice of the appropriate chiral column, eluent, and conditions necessary for effective separation of stereoisomers, such as a pair of enantiomers, is well known to one of ordinary skill in the art (e.g., Jacques et al., Enantiomers, Racemates, and Resolutions, John Wiley and Sons, Inc., 1981).

As used herein, the term “pharmaceutically acceptable” refers to compounds, materials, compositions, or formulations which are, within the scope of sound medical judgment, suitable for use in contact with the tissues of human beings and non-human animals without excessive toxicity, irritation, allergic response, or other problems or complications that commensurate with a reasonable benefit/risk ratio, in accordance with the guidelines of regulatory agencies of a certain country, such as the Food and Drug Administration (FDA) in the United States or its corresponding agencies in countries other than the United States (e.g., the European Medicines Agency (EMA) in Europe, the National Medical Products Administration (NMPA) in China).

As used herein, the term “salt” refers to acid or base salts of the original compound. In some cases, the salt is formed in situ during preparation of the original compound, i.e., the designated synthetic chemistry procedures produce the salt instead of the original compound. In some cases, the salt is obtained via modification of the original compound. In some cases, the salt is obtained via ion exchange with an existing salt of the original compound. Examples of salts include, but are not limited to, mineral or organic acid salts of basic residues such as amines, as well as alkali or organic salts of acidic residues such as carboxylic acids and phosphonic acids. For original compounds containing a basic residue, the salts can be prepared by treating the compounds with an appropriate amount of a non-toxic inorganic or organic acid; alternatively, the salts can be formed in situ during preparation of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the salts can be prepared by treating the compounds with an appropriate amount of a non-toxic base; alternatively, the salts can be formed in situ during preparation of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Optionally, the salts can be prepared by reacting the free acid or base form of the original compounds with a stoichiometric amount or more of an appropriate base or acid, respectively, in water or an aqueous solution, an organic solvent or an organic solution, or a mixture thereof. Lists of exemplary pharmaceutically acceptable salts can be found in Remington's Pharmaceutical Sciences, 23rd Ed., Elseiver, 2021 as well as Handbook of Pharmaceutical Salts: Properties, Selection, and Use, 2^{nd} revised edition, Stahl and Wermuth, Eds., Wiley-VCH, Weinheim, 2011.

As used herein, the term “excipient” refers to any components present in the pharmaceutical formulations disclosed herein, other than the active ingredient (i.e., a compound or composition of the present disclosure).

As used herein, the term “effective amount” of a material refers to a nontoxic but sufficient amount of the material to provide the desired result (e.g., reduced inflammation or reduced mature mast cell number). The exact amount required may vary from subject to subject, depending on the species, age, and general condition of the subject, the severity of the condition, disorder, or disease that is being treated, the active ingredient or therapy used, and the like.

### EXAMPLES

The examples below describe studies to evaluate the activities of exemplary compounds BK40143, BK40195, BK40196, and BK40197, whose structures are shown below.

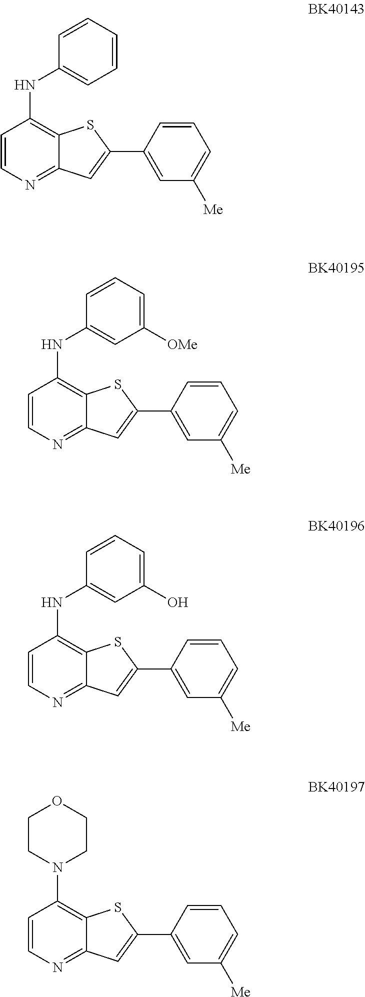 [SMILES: Cc1cccc(-c2cc3nccc(Nc4ccccc4)c3s2)c1 ; COc1cccc(Nc2ccnc3cc(-c4cccc(C)c4)sc23)c1 ; Cc1cccc(-c2cc3nccc(Nc4cccc(O)c4)c3s2)c1 ; Cc1cccc(-c2cc3nccc(N4CCOCC4)c3s2)c1]

The synthetic methods for the tested compounds are disclosed in WO2021/155195, which is incorporated herein by reference. The methods are compatible with a wide variety of functional groups and starting materials. Thus, a wide variety of compounds can be obtained from the disclosed methods.

### Example 1. Identification of Activity Against c-Kit

### A. Materials and Methods

Kinase assays were performed using KINASESEEKER™ (Luceome Biotechnologies, LLC; Tucson, AZ). KINASESEEKER™ is a homogeneous competition binding assay in which the displacement of an active site-dependent probe by an inhibitor is measured by a change in luminescence signal. Luminescence readout translates into a highly sensitive and robust assay with low background and minimal interference from test compounds. See J. Am. Chem. Soc., 2010, 132, 11727-11735.

Stock solutions of compounds were serially diluted in DMSO to make assay stocks. Prior to initiating IC₅₀ determination, the test compounds were evaluated for false positives against split-luciferase. The compounds were screened against kinases at a minimum of 8 different concentrations in duplicate. For kinase assays, a 24 μL aliquot of lysate containing Cfluc-kinase and Fos-Nfluc was incubated for 2 hours with either 1 μL of DMSO (for no-inhibitor control) or compound solution in DMSO in presence of a kinase specific probe. 80 μL of luciferin assay reagent was added to each solution and luminescence was immediately measured on a luminometer.

The % Inhibition and % Activity Remaining was calculated using the following equation:

$$\% Inhibition = ( {ALU}_{Control} - {ALU}_{Sample} ) / {ALU}_{Control} \times 100$$

$$\% Activity Remaining = 100 - \% Inhibition$$

The % Activity Remaining was plotted against compound concentration and the IC₅₀ was determined for each compound using a minimum 8-point curve.

### B. Results

The IC₅₀ values of BK40143, BK40195, and BK40196 against DDR1, DDR2, and c-kit are shown in Table 1. These compounds, previously considered DDR inhibitors, exhibited a much higher activity against c-kit.

TABLE 1
Summary of IC₅₀ values against DDR1, DDR2, and c-kit

| | DDR1 IC₅₀ (μM) | DDR2 IC₅₀ (μM) | c-kit IC₅₀ (μM) |
|---|---|---|---|
| BK40143 | 7.05 ± 1.05 | 6.03 ± 0.93 | 0.75 ± 0.11 |
| BK40195 | 2.20 ± 0.26 | 1.90 ± 0.26 | 0.288 ± 0.023 |
| BK40196 | 1.07 ± 0.13 | 0.922 ± 0.130 | 0.276 ± 0.035 |

### Example 2. In Vitro Studies of the Effect of c-Kit Inhibition on MC Maturation

### A. Materials and Methods

A human mast cell (MC) line that arose spontaneously from a culture of non-transformed hematopoietic progenitor cells is described in J Allergy Clin Immunol, 2011, 127:815-22, which is incorporated herein by reference. The cells, termed LUVA cells, are an immortalized human MC line that can be maintained without stem cell factor (SCF) and that display high levels of normally signaling c-kit and FceR1.

The LUVA cells (Kerafast, EG1701-FP) were cultured in StemPro-34 serum-free media with 10% nutrient supplement, 1% L-glutamine, and 1% pen/strep and grown under normal atmospheric conditions (5% CO₂). The cells were plated and treated with 100 ng/mL of SCF overnight and then treated with DMSO or an ascending dose of either BK40143, BK40196, or BK40197 for 48 hours, before collection and incubation with two flow-conjugated antibodies (PE anti-mouse CD117 (c-kit) and APC/fire 750 anti-mouse FceR1a) and subsequent analysis via flow cytometry. Group differences were compared using unpaired Student's t-tests.

### B. Results

No differences were detected in cell proliferation of the LUVA cells in the presence or absence of SCF. Further, the LUVA cells displayed high levels of c-kit (detected by the CD117 antibody) and FceR1 (detected by the anti-mouse FceR1a antibody) as expected. The presence of high levels of c-kit (activation) and FceR1 signifies that the LUVA cells were mature MCs.

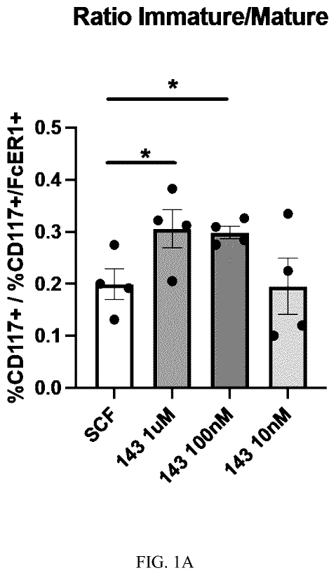

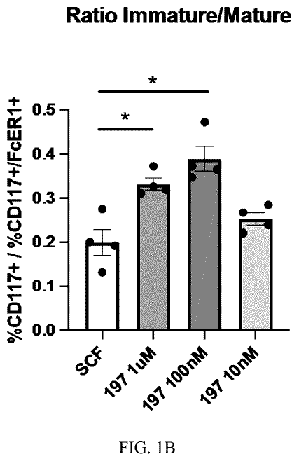

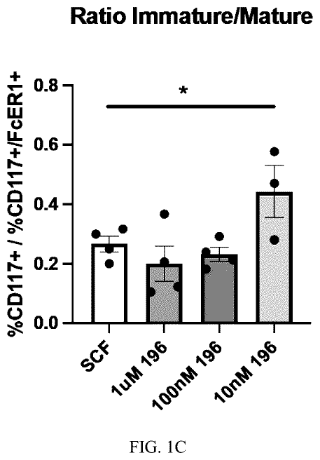

FIGS. 1A-C show that inhibition of c-kit with BK40143, BK40197, or BK40196 significantly increased the number of immature MCs (expressing CD117 only) compared to mature MCs (expressing both CD117 and FceR1), suggesting that c-kit inhibition on these MCs can prevent the maturation of MCs and therefore prevent exacerbation of immune responses. These data demonstrate that c-kit inhibition by these compounds can prevent or mitigate an inflammatory reaction via blockade of MCs maturation and subsequent degranulation that would release inflammatory factors that trigger an immune response.

### Example 3. In Vivo Studies of the Effect of c-Kit Inhibition on MC Maturation

### A. Materials and Methods

Wild-type C57BL/6 mice were dosed with BK40197 via intraperitoneal injection at 50 mg/kg (maximum tolerated dose) per day for five days before blood samples were taken and peripheral mast cells were isolated via flow cytometry. Cell gating was performed using antibodies against specific epitopes for cells: CD45 antibody for leukocytes, FceR1 antibody for mature mast cells, and CD117 antibody for immature and mature mast cells.

### B. Results

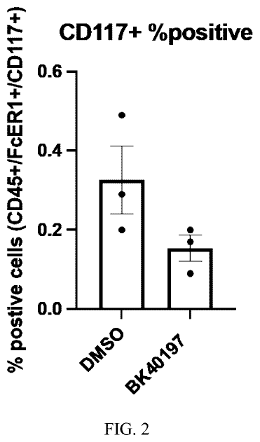

As shown in FIG. 2, gating for mature MCs, i.e., CD117+/FceR1+, was reduced by 48% (p=0.06, Student's t-test) in the BK40197 treatment group, compared to the negative control (DMSO). This result is consistent with the LUVA cell data from Example 2. Collectively, Examples 2 and 3 demonstrate that inhibition of c-kit by the c-kit inhibitors can mitigate maturation of MCs to prevent an inflammatory reaction.

### Example 4. Studies of MC Maturation in Different Mouse Models

### A. Materials and Methods

To determine whether there is any difference in MC maturation or proliferation between a wild-type animal and various transgenic models of human disease, 12-month-old wild-type C57BL/6 mice were compared to transgenic APP mice (expressing triple mutants of the human amyloid precursor protein in vascular angiopathies and Alzheimer's disease), tg4510 mice (expressing the human P301L mutation of tau in frontotemporal dementia) and A53 mice (expressing the human A53T mutation of alpha-synuclein in Parkinson's disease). Blood samples were collected and analyzed with flow cytometry. Specifically, the samples were treated with three flow-conjugated antibodies (CD45 antibody for leukocytes, FceR1 antibody for mature mast cells, CD117 antibody for immature and mature mast cells) and analyzed for maturation of mast cell progenitors (CD45+/CD117+) to mature mast cells (CD45+/CD117+/FceR1+). Group differences were compared using an ordinary one-way ANOVA test.

### B. Results

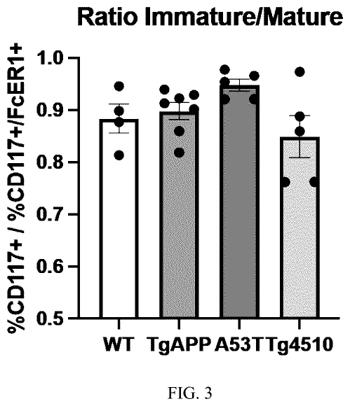

As shown in FIG. 3, there was no difference in MC maturation between the tested strains and genotypes of mice, suggesting that all of them are suitable for testing the effects of the c-kit inhibitors on MC maturation.

### Example 5. Studies of the Effect of c-Kit Inhibition by BK40143 on MC Maturation in Transgenic APP Mice

### A. Materials and Methods

12-month-old transgenic APP mice were injected intraperitoneally with either 5 mg/kg BK40143 or DMSO for six weeks before whole-blood samples were collected into tubes with 1000 U/mL heparin. Blood samples were then treated with the three flow-conjugated antibodies and analyzed for maturation of mast cell progenitors to mature mast cells, as described in Examples 3 and 4. Group differences were compared using an unpaired Student's t-test.

### B. Results

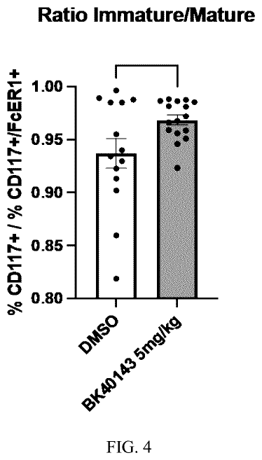

As shown by FIG. 4, inhibition of c-kit by BK40143 (5 mg/kg) significantly increased the ratio of immature MCs (expressing CD117 only) to mature MCs (expressing both CD117 and FceR1).

### Example 6. Studies of the Effect of c-Kit Inhibition by BK40195 on MC Maturation in Transgenic APP Mice

### A. Materials and Methods

12-month-old transgenic APP mice were injected intraperitoneally with either 25 mg/kg BK40195 or DMSO for six weeks before having whole-blood samples collected into tubes containing 1000 U/mL heparin. Blood samples were then treated with the three flow-conjugated antibodies and analyzed for maturation of mast cell progenitors to mature mast cells, as described in Example 5. Differences were compared using an unpaired Student's t-test.

### B. Results

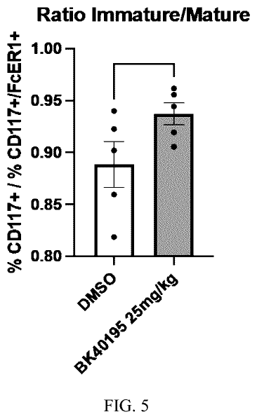

As shown by FIG. 5, inhibition of c-kit by BK40195 (25 mg/kg) significantly increased the ratio of immature MCs (expressing CD117 only) to mature MCs (expressing both CD117 and FceR1).

### Example 7. Studies of the Effect of c-Kit Inhibition by BK40197 on MC Maturation in Transgenic APP Mice

### A. Materials and Methods

12-month-old transgenic APP mice were injected intraperitoneally with either 45 mg/kg BK40197 or DMSO for six weeks before having whole-blood samples collected into tubes containing 1000 U/mL heparin. Blood samples were then treated with the three flow-conjugated antibodies and analyzed for maturation of mast cell progenitors to mature mast cells, as described in Example 5. Differences were compared using an unpaired Student's t-test.

### B. Results

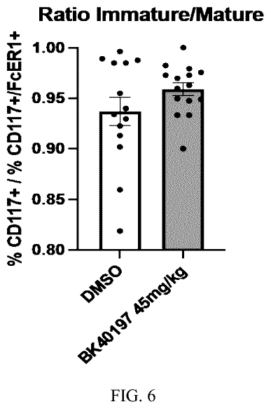

As shown by FIG. 6, inhibition of c-kit by BK40197 (45 mg/kg) significantly increased the ratio of immature MCs (expressing CD117 only) to mature MCs (expressing both CD117 and FceR1).

### Example 8. Studies of the Effect of c-Kit Inhibition by BK40196 on MC Maturation in the Spleen of Transgenic APP Mice

### A. Materials and Methods

The studies in this example compared the number of immature MCs to the number of mature MCs in the spleen.

12-month-old transgenic APP mice were injected intraperitoneally with 20 mg/kg BK40196, 40 mg/kg BK40196, or DMSO for six weeks before having spleens collected for analysis. Spleen samples were then treated with the three flow-conjugated antibodies and analyzed for maturation of mast cell progenitors to mature mast cells, as described in Example 5. Differences were compared using an unpaired Student's t-test.

### B. Results

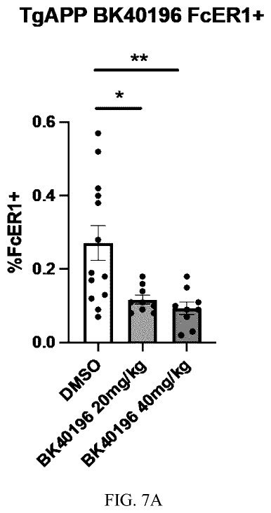

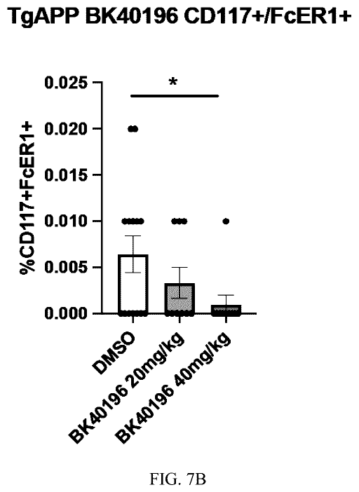

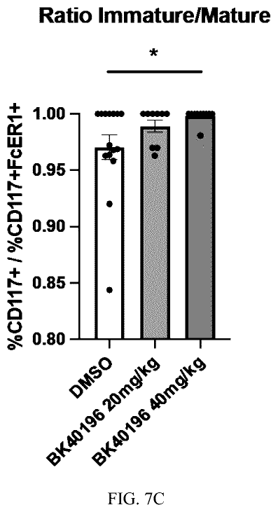

As shown by FIG. 7A, inhibition of c-kit by BK40196 significantly reduced the total number of mature MCs (FceR1+) in the spleen in a dose-dependent manner. Similarly, as shown in FIG. 7B, inhibition of c-kit by BK40196 significantly reduced the total number of mature MCs (CD117+/FceR1+) in the spleen in a dose-dependent manner.

As shown by FIG. 7C, BK40196 increased the ratio of immature MCs (expressing CD117 only) to mature MCs (expressing both CD117 and FceR1) in the spleen, suggesting that BK40196 can prevent the maturation of MCs and therefore prevent exacerbation of immune responses.

The spleen data are consistent with the blood data shown in the other examples and provides an additional line of powerful evidence demonstrating that the transition from immature to mature MCs results from c-kit.

Collectively, the data from the foregoing examples show that BK40143, BK40195, BK40196, and BK40197 inhibited c-kit and reduced the ratio of immature mast cells to mature mast cells, thereby preventing mast cell activation. In vivo experiments showed that BK40143, BK40195, and BK40197 increased the number of immature mast cells in the blood circulation of mice and reduced the number of mature mast cells, signifying that c-kit inhibition can prevent detrimental activation of mast cells. Moreover, in vivo experiments also show that BK40196 significantly reduced the number of mature cells relative to immature cells in the spleen of mice.

### Example 9. Kinase Binding of BK40143, BK40195, BK40196 and BK40197

### A. Materials and Methods

The KINOMEscan™ screening platform (Eurofins DiscoverX Corporation, San Diego, USA) was performed using an active site-directed competition binding assay to quantitatively measure interactions between BK40143, BK40197, BK40195, and BK40196 and more than 450 human kinases and disease relevant mutant variants. KINOMEscan™ assays do not require ATP and thereby report true thermodynamic interaction affinities, as opposed to IC₅₀ values, which can depend on ATP concentration (https://www.eurofinsdiscovery.com/solution/scanmax). BK40143, BK40197, BK40195, and BK40196 that bind the kinase active site and directly (sterically) or indirectly (allosterically) prevent kinase binding to an immobilized, proprietary Eurofins ligand, and reduce the amount of kinase captured on a solid support, whereas BK40143 and BK40197, BK40195, and BK40196 that do not bind the kinase have no effect on the amount of kinase captured. Screening “hits” were identified by measuring the amount of kinase captured in test (BK40143, BK40197, BK40195, and BK40196) versus control samples by using a quantitative and ultra-sensitive qPCR method that detects the associated DNA label. BK40143, BK40197, BK40195, and BK40196 were screened at 10 μM concentration, and results for primary screen binding interactions were reported as ‘% Ctrl’ (POC), where lower numbers indicate stronger hits and calculated as follows:

$$( \frac{test compound signal - positive control signal}{negative compound signal - positive control signal} ) \times 100$$

test compound=BK40143 or BK40197 or BK40195 or BK40196
negative control=DMSO (100% Ctrl)
positive control=control compound (0% Ctrl)

Based on screening data of profiled compounds, a proportional relationship between primary screening results and corresponding compound/target affinities can be described as binding constants (K_{d} values) for the indicated ranges of percent of control (PoC) values, with tighter binding (higher affinity) interactions associated with lower PoC values, and weaker binding (lower affinity) associated with higher PoC values. Briefly, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 m) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 100× stocks in 100% DMSO and directly diluted into the assay. All reactions were performed in polypropylene 384-well plates in a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### B. Results

The binding interactions against 450 human kinases and disease relevant mutant variants were quantitatively measured using KINOMEscan. As shown in Table 2, primary screening results and corresponding compound/target affinities indicated PoC values with tighter binding (higher affinity) was associated with lower PoC values and weaker binding (lower affinity) was associated with higher PoC values. Decreases in ligand binding to human c_KIT and related c-KIT mutants were observed for BK41043, BK40195, BK40196 and BK40197, with a PoC value of zero for any of the test compounds corresponding to completely abolishing ligand binding (100% inhibition) of human c-KIT and related mutants. Ligand binding of SRC, SYK, and FYN, which are related to mast cell activation, was also decreased by BK41043, BK40195, BK40196 and BK40197. The lowest range of POC 0≤x<0.1 strongly prevents kinase binding associated with mast activity and inflammation.

TABLE 2
Screening of kinase binding of BK40143, BK40195, BK40196 and BK40197.

| | % Ctrl @ 10 μM | % Ctrl @ 10 μM | % Ctrl @ 10 μM | % Ctrl @ 10 μM |
|---|---|---|---|---|
| | BK40143 | BK40195 | BK40196 | BK40197 |
| Target | (% inhibition) | (% inhibition) | (% inhibition) | (% inhibition) |
| KIT | 0 (100) | 0 (100) | 0 (100) | 0.3 (97.7) |
| KIT (A829P) | 52 (48) | 6.3 (93.7) | 21 (79) | 100 (0) |
| KIT (D816H) | 46 (54) | 6.1 (93.9) | 3.9 (94.1) | 100 (0) |
| KIT (D816V) | 35 (65) | 1.7 (98.3) | 0.2 (99.8) | 67 (33) |
| KIT (L576P) | 3.5 (96.5) | 8.1 (91.9) | 0.8 (99.2) | 1.8 (98.2) |
| KIT (V559D) | 0.25 (99.75) | 0.2 (99.8) | 0 (100) | 0.35 (99.65) |
| KIT (V559D, T670I) | 19 (81) | 2.5 (97.5) | 2.4 (97.6) | 20 (80) |
| KIT (V559D, V654A) | 18 (82) | 2.5 (97.5) | 1.3 (98.7) | 21 (79) |
| KIT autoinhibited | 69 (31) | 22 (78) | 56 (44) | 52 (48) |
| SRC | 5.3 (94.7) | 0.35 (99.65) | 0.1 (99.9) | 81 (19) |
| SYK | 5.5 (94.5) | 12 (88) | 2.3 (97.7) | 44 (56) |
| FYN | 10 (90) | 2.4 (97.6) | 0 (100) | 56 (44) |

### Example 10. In Vivo Studies of BK40143, BK40197, BK40195 and BK4096 in Mouse Models of Mast Cell Activation

### A. Materials and Methods

Wild type C57BL/6J mice were treated with C48/40 to induce mast cell activation. Compound 48/80 is a polymer produced by the condensation of N-methyl-p-methoxyphenethylamine with formaldehyde. It promotes histamine release, and in biochemical research, compound 48/80 (for example, Catalog No. C2313, Sigma-Aldrich, St. Louis, MO) is used to promote mast cell degranulation. Male and female, 4-8 months old wild type C57BL/6 mice were treated only once with intraperitoneal (I.P.) injection of DMSO (control) or DMSO for 2 hours followed by C48/40 for 0.5-1.5 hours. Mice were injected with DMSO approximately 2 hours prior to injection (IP) with 50 μg C48/40 to show degranulation of mast cells 0.5-1.5 hours post-injection of C48/40. Animals were also injected with 20 mg/kg BK40197, BK40195 or BK40196; or 10 mg/kg BK40143 approximately two hours before I.P. injection of C48/40 to show the effects of the drugs on mast cell degranulation approximately 0.5-1.5 hours post-injection of C48/40. Drugs and C48/40 were injected on the same site. n=6 per treatment. Blood was collected and a smear was prepared and stained with Toluidine blue to show mast cell morphology.

### B. Results

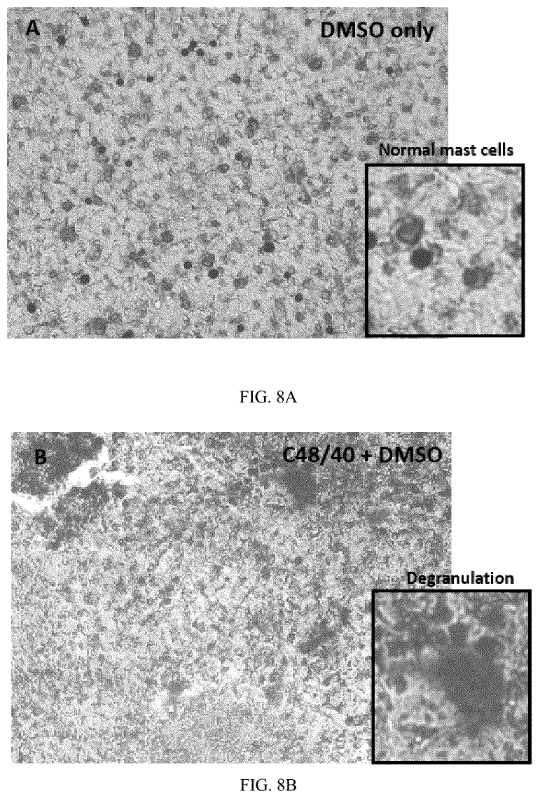

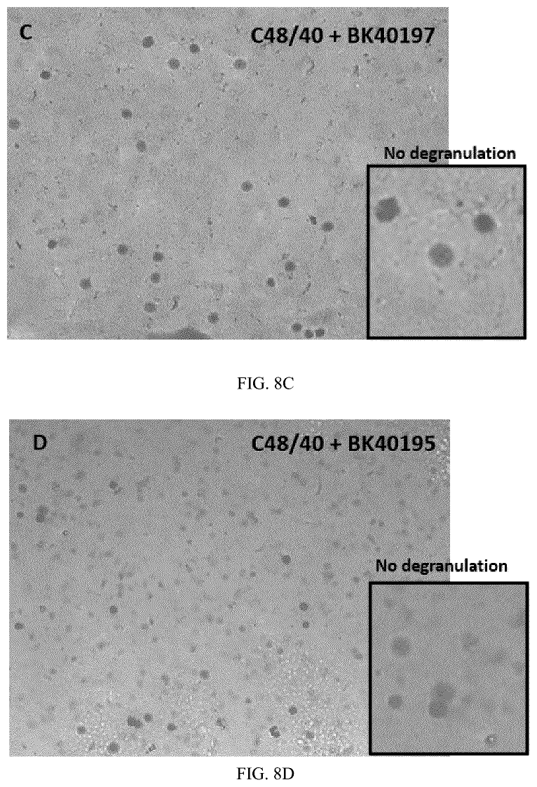

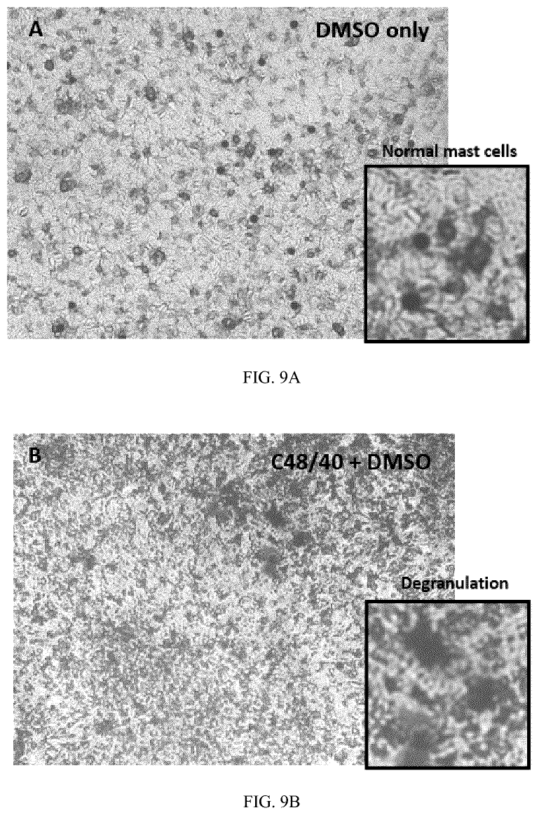

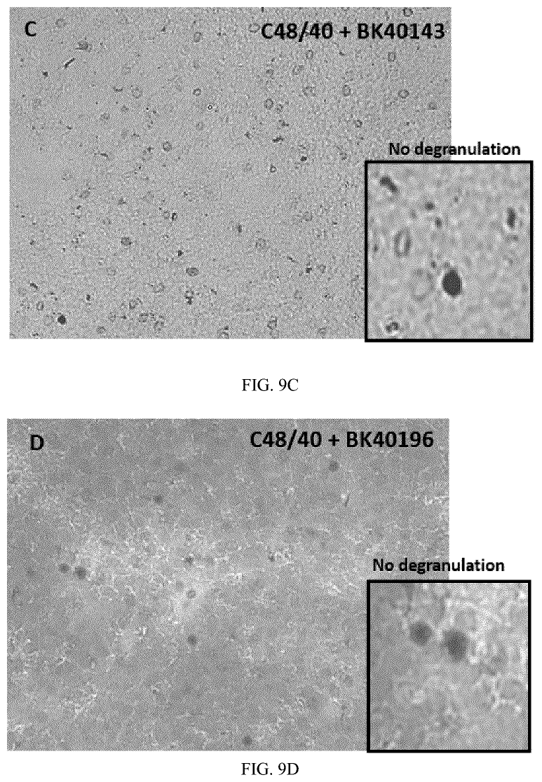

As shown in FIGS. 8B and 9B, toluidine blue staining of blood smear showed that Compound 48/80 successfully induced mast cell degranulation only a short time (approximately less than 2 hours after I.P injection) compared to control (DMSO) mice. However, a single injection of either BK40197, BK40195, BK40143, BK40196 (FIGS. 8C, 8D, 9C, and 9D, respectively) two hours prior to C48/40 treatment, significantly prevented mast cell degranulation, indicating these compounds have strong anti-inflammatory effects via mitigation of mast cell activation.

What is claimed is:

1. A method of treating an inflammatory condition or preventing a flare in the inflammatory condition in a subject in need thereof, comprising administering to the subject an effective amount of a compound having the following formula or a pharmaceutically acceptable salt, hydrate, or hydrated salt thereof: 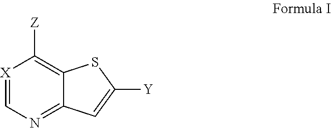
wherein X is N or CH;
wherein Y is C₆₋₁₀ aryl unsubstituted or substituted with R¹, C₆₋₁₀ aryl unsubstituted or substituted with R¹, C₅₋₁₀ heteroaryl unsubstituted or substituted with R¹, or N-methylpiperazinyl;
wherein R¹ is —CF₃, —(CH₂)ₙ—R², —(CH₂)ₙ—C(O)—R², or —O(CH₂)ₙ—R²;
wherein R² is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl;
wherein Z is heteroaryl, heterocyclyl, or NR³R⁴;
wherein R³ and R⁴ are independently H, C₁₋₃ alkyl, C₁₋₃ alkoxy, or phenyl unsubstituted or substituted with R¹⁰;
wherein R¹⁰ is halogen, —CN, hydroxyl, —CF₃, C₁₋₃ alkyl, C₁₋₃ alkoxy, amino, C₁₋₃ alkyl amino, or di C₁₋₃ alkyl amino; and
wherein n is an integer selected from 0 to 3.

2. The method of claim 1, wherein X is CH.

3. The method of claim 1, wherein Y is phenyl substituted with R¹.

4. The method of claim 1, wherein R¹ is —(CH₂)ₙ—R².

5. The method of claim 1, wherein R² is H.

6. The method of claim 1, wherein n is 1.

7. The method of claim 1, wherein Z is heterocyclyl.

8. The method of claim 7, wherein Z is morpholin-1-yl.

9. The method of claim 1, wherein Z is NR³R⁴.

10. The method of claim 9, wherein Z is NHR⁴.

11. The method of claim 9, wherein R⁴ is phenyl unsubstituted or substituted with R¹⁰.

12. The method of claim 11, wherein R⁴ is phenyl substituted with R¹⁰, wherein R¹⁰ is hydroxyl or methoxy.

13. The method of claim 1, wherein X is CH, Y is phenyl unsubstituted or substituted with R¹, and Z is heterocyclyl.

14. The method of claim 13, wherein Y is phenyl substituted with R¹ in the meta position and Z is morpholin-1-yl.

15. The method of claim 13, wherein R¹ is —CH₃.

16. The method of claim 13, wherein the compound is 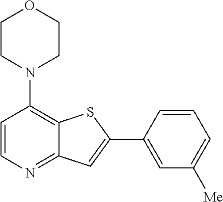 [SMILES: Cc1cccc(-c2cc3nccc(N4CCOCC4)c3s2)c1]
or a pharmaceutically acceptable salt, hydrate, or hydrated salt thereof.

17. The method of claim 1, wherein X is CH, Y is phenyl substituted with R¹, and Z is NR³R⁴.

18. The method of claim 17, wherein Y is phenyl substituted with R¹ in the meta position and Z is NHR⁴.

19. The method of claim 17, wherein R¹ is —CH₃ and R⁴ is phenyl, 3-hydroxyphenyl, or 3-methoxyphenyl.

20. The method of claim 17, wherein the compound is 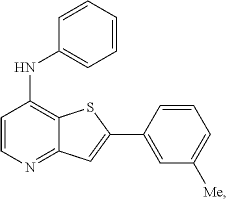 [SMILES: Cc1cccc(-c2cc3nccc(Nc4ccccc4)c3s2)c1]
or a pharmaceutically acceptable salt, hydrate, or hydrated salt thereof.

21. The method of claim 1, wherein the effective amount of the compound inhibits c-kit.

22. The method of claim 1, wherein the effective amount of the compound increases immature mast cell number or increases a ratio of immature mast cells to mature mast cells in a tissue, organ, or system of the subject, as compared to the corresponding number or ratio before administrating or in the absence of the effective amount of the compound.

23. The method of claim 1, wherein the inflammatory condition is present in a tissue, joint, organ, or system of the subject, selected from the group consisting of skin, connective tissues, mucosal tissues, organs, cardiovascular system, lymphatic system, skeletal system, respiratory system, and digestive system.

24. The method of claim 1, wherein the inflammatory condition is related to c-kit upregulation or hyperactivity.

25. The method of claim 1, wherein the inflammatory condition is related to an overabundance of mature mast cells.

26. The method of claim 1, wherein the inflammatory condition is selected from the group consisting of mastocytosis, mast cell activation syndrome, hereditary alpha tryptasemia, urticaria, Lyme disease, mast cell leukemia, chronic obstructive pulmonary disease, long COVID, asthma, inflammatory bowel disease, arthritis, and gout.

27. The method of claim 1, wherein the compound is administered systemically, locally, or by inhalation.

28. The method of claim 1, further comprising administering a second therapeutic agent.

29. The method of claim 28, wherein the second therapeutic agent is selected from the group consisting of non-steroidal anti-inflammatory agents, steroids, bronchodilators, and biologics.
